(19) United States
(12) Patent Application Publication
(10) Pub. No.: US 2013/0123131 A1
(43) Pub. Date: May 16, 2013

(54) Process for Ensuring Consistency and Reproducibility of a Diagnostic or Research Method

(71) Applicant: Nodality, Inc., South San Francisco, CA (US)

(72) Inventors: Norman Purvis, Franklin, TN (US); Santosh Putta, Foster City, CA (US); Matt Westfall, Burlingame, CA (US); David Rosen, Mountain View, CA (US); David Spellmeyer, Oakland, CA (US); Jason Ptacek, Redwood City, CA (US)

(73) Assignee: Nodality, Inc., South San Francisco, CA (US)

(21) Appl. No.: 13/673,213

(22) Filed: Nov. 9, 2012

Related U.S. Application Data
(60) Provisional application No. 61/557,831, filed on Nov. 9, 2011.

(51) Int. Cl. C12Q 1/02; G01N 33/68

(52) U.S. Cl. CPC C12Q 1/025; G01N 33/68
USPC 506/9; 506/10; 506/18

(57) ABSTRACT

A method is disclosed in which control processes are used to maintain consistency across a research or diagnostic series of steps. Some embodiments of the processes include the use of fresh or lyophilized cell lines, beads, surface or other markers. The use of quality control processes is intended to monitor data from the underlying methods in order to detect unacceptable variations and to allow for exclusion or normalization. Overlapping control processes allows for tighter control and for redundancy in the monitoring.

### CROSS-REFERENCE

This application claims the benefit of U.S. Provisional Application No. 61/557,831 filed Nov. 9, 2011, which application is incorporated herein by reference.

### BACKGROUND OF THE INVENTION

Methods for diagnosis, monitoring disease, or prognosis are useful to health care practitioners. In a diagnostic or laboratory setting, it is useful to ensure that there is a process to ensure quality in an important health care related method.

### SUMMARY OF THE INVENTION

One embodiment of the present invention is a functional, quality control method for flow cytometry or mass spectrometry. It can be used used in diagnosis, prognosis, monitoring therapy, or drug development or other regulated tests and it comprising the steps of providing a microtiter plate; distributing sample cells into wells of the microtiter plate; distributing standard cells into wells of the microtiter plate; distributing rainbow control particles (RCPs) into wells of the microtiter plate; contacting the standard cells and sample cells with at least one modulator; measuring one or more activatable elements and surface markers in the standard cells by flow cytometry or mass spectrometry to create standard cell data; measuring one or more activatable elements in the sample cells by flow cytometry or mass spectrometry to create test data; measuring RCPs by flow cytometry or mass spectrometry to create RCP data; comparing the standard cell data and RCP data to a preset range of acceptable values; and optionally normalizing or excluding the test data based on the standard cell data or RCP data.

One embodiment of the present invention comprises measuring at least two activatable elements in standard cells to create standard cell data; comparing the standard cell data to a preset range of acceptable values; measuring at least two activatable elements in sample cells to create test data; and excluding or normalizing test data when the standard cell data is not within the preset range. The method further comprises providing one or more microtiter plates; distributing live, sample cells into wells of a microtiter plate; distributing live, standard cells into wells of another microtiter plate; measuring at least two activatable elements in the standard cells to create standard cell data; measuring at least two activatable elements in the sample cells when the standard cell data is within the preset range. The method can use standard cells like stable cell lines, two examples are GDM-1 or RS;411.

The method can also include providing a plurality of quality controls including the addition of beads, addition of cell lines, addition of stain controls, and the monitoring of cell surface or intracellular markers. Additionally, one aspect of the invention involves monitoring each step and the time it was performed and placing quality control data into a database for future reference.

The invention also includes a kit comprising two or more reagents, compounds or other devices selected from the group of: live cell lines, lyophilized cells, RCPs; and three or more reagents selected from the group of: antibodies directed to cell surface markers, antibodies directed to internal cell markers, modulators, buffers, fixatives, binding elements, and permeabilizers, The kit may additionally comprise a cytometric capture array, buffers and reagents.

One embodiment of the invention is a functional, quality control method for use when analyzing samples with flow cytometry or mass spectrometry, comprising the steps of: providing a holder having wells; distributing sample cells into wells; adding one or more reagents to wells, the reagents produce a consistent result under the same conditions used for the sample cells, the reagents include at least one or more of: standard cells, rainbow control particles (RCPs), and surface marker detection compounds; contacting the sample cells and reagents with at least one modulator; processing the reagents to obtain quality control data; measuring at least two activatable elements in the sample cells to create test data; comparing the quality control data to a preset range of acceptable values; and analyzing the test data when the quality control data is within a preset range.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a flow chart of steps of one embodiment along with where some monitoring steps can be placed.

FIG. 2 shows a table with the performance of two cell lines across multiple modulators, times, and other variables.

FIG. 3 shows the performance of the GDM-1 cell line across multiple runs. Each line represents a separate node that was measured.

FIG. 4 shows the performance of the RS4;11 cell line across multiple runs. Each line represents a separate node that was measured.

FIG. 5 shows the performance of normal surface markers indicated by the solid circles and surface markers for apoptosing cells indicated by the open circles.

FIG. 6 shows the cell line monitoring of the GDM-1 cell line within a specific study.

FIG. 7 shows the cell line monitoring of the RS4;11 cell line within a specific study.

FIG. 8 shows the performance of GM13023 BRCA2 mutated cells with or without Flow Count Beads combined in the same well.

FIG. 9 shows the performance of GM13023 BRCA2 mutated cells with or without Flow Count Beads combined in the same well.

### DETAILED DESCRIPTION OF THE INVENTION

The present methods incorporate information disclosed in other applications and texts. The following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

Also, patents and applications that are incorporated by reference include U.S. Pat. Nos. 7,381,535, 7,393,656, 7,563,584, 7,695,924, 7,695,926, 7,939,278, 8,148,094, 8,187,885, 8,198,037, 8,206,939, 8,214,157, 8,227,202, 8,242,248; U.S. Ser. Nos. 11/338,957, 11/655,789, 12/061,565, 12/125,759, 12/125,763, 12/229,476, 12/432,239, 12/432,720, 12/471,158, 12/501,274, 12/501,295, 12/538,643, 12/551,333, 12/581,536, 12/606,869, 12/617,438, 12/687,873, 12/688,851, 12/703,741, 12/713,165, 12/730,170, 12/778,847, 12/784,478, 12/877,998, 12/910,769, 13/082,306, 13/091,971, 13/094,731, 13/094,735, 13/094,737, 13/098,902, 13/098,923, 13/098,932, 13/098,939, 13/384,181; 13/645,325; International Applications Nos. PCT/US2011/001565, PCT/US2011/065675, PCT/US2011/026117, PCT/US2011/029845, PCT/US2011/048332; and U.S. Provisional Applications Ser. Nos. 60/304,434, 60/310,141, 60/646,757, 60/787,908, 60/957,160, 61/048,657, 61/048,886, 61/048,920, 61/055,362, 61/079,537, 61/079,551, 61/079,579, 61/079,766, 61/085,789, 61/087,555, 61/104,666, 61/106,462, 61/108,803, 61/113,823, 61/120,320, 61/144,68, 61/144,955, 61/146,276, 61/151,387, 61/153,627, 61/155,373, 61/156,754, 61/157,900, 61/162,598, 61/162,673, 61/170,348, 61/176,420, 61/177,935, 61/181,211, 61/182,518, 61/182,638, 61/186,619, 61/216,825, 61/218,718, 61/226,878, 61/236,281, 61/240,193, 61/240,613, 61/241,773, 61/245,000, 61/254,131, 61/263,281, 61/265,585, 61/265,743, 61/306,665, 61/306,872, 61/307,829, 61/317,187, 61/327,347, 61/350,864, 61/353,155, 61/373,199, 61/374,613, 61/381,067, 61/382,793, 61/423,918, 61/436,534, 61/440,523, 61/469,812, 61/499,127, 61/515,660, 61/521,221, 61/542,910, 61/557,831, 61/558,343, 61/565,391, 61/565,929, 61/565,935, 61/591,122, 61/640,794, 61/658,092, 61/664,426, 61/693,429, and 61/713,260.

Some commercial reagents, protocols, software and instruments that are useful in some embodiments of the present invention are available from Becton Dickinson and Beckman Coulter see their websites. Relevant articles include High-content single-cell drug screening with phosphospecific flow cytometry, Krutzik et al., Nature Chemical Biology, 23 Dec. 2007; Irish et al., FLt3 ligand Y591 duplication and Bcl-2 over expression are detected in acute myeloid leukemia cells with high levels of phosphorylated wild-type p53, Neoplasia, 2007; Irish et al. Mapping normal and cancer cell signaling networks: towards single-cell proteomics, Nature, Vol. 6 146-155, 2006; Irish et al., Single cell profiling of potentiated phospho-protein networks in cancer cells, Cell, Vol. 118, 1-20 July 23, 2004; Schulz, K. R., et al., Single-cell phospho-protein analysis by flow cytometry, Curr Protoc Immunol, 2007, 78:8 8.17.1-20; Krutzik, P. O., et al., Coordinate analysis of murine immune cell surface markers and intracellular phosphoproteins by flow cytometry, J Immunol. 2005 Aug. 15;175(4):2357-65; Krutzik, P.O., et al., Characterization of the murine immunological signaling network with phosphospecific flow cytometry, J Immunol. 2005 Aug. 15;175(4):2366-73; Shulz et al., Current Protocols in Immunology 2007, 78:8.17.1-20; Stelzer et al., Use of Multiparameter Flow Cytometry and Immunophenotyping for the Diagnosis and Classification of Acute Myeloid Leukemia, Immunophenotyping, Wiley, 2000; and Krutzik, P.O. and Nolan, G. P., Intracellular phospho-protein labeling techniques for flow cytometry: monitoring single cell signaling events, Cytometry A. 2003 October;55(2):61-70; Hanahan D., Weinberg, The Hallmarks of Cancer, Cell, 2000 Jan. 7;100(1) 57-70; and Krutzik et al, High content single cell drug screening with phophosphospecific flow cytometry, Nat Chem Biol. 2008 February;4(2):132-42. Experimental and process protocols and other helpful information can be found at http:/proteomics.stanford.edu. The articles and other references cited below are also incorporated by reference in their entireties for all purposes. More specific procedures can be found in the following manuscripts: Rosen D B, Putta S, Covey T et al. Distinct Patterns of DNA Damage Response and Apoptosis Correlate with Jak/Stat and PI3Kinase Response Profiles in Human Acute Myelogenous Leukemia. 2010. PLoS ONE. 5 (8): e12405; Kornblau S M, Minden M D, Rosen D B, Putta S, Cohen A, Covey T, et al., Dynamic Single-Cell Network Profiles in Acute Myelogenous Leukemia Are Associated with Patient Response to Standard Induction Therapy. 2010. Clinical Cancer Research. 16 (14): 3721-33 Jan 31; Rosen DB et al., Functional Characterization of FLT3 Receptor Signaling Deregulation in AML by Single Cell Network Profiling (SCNP). 2010. PLoS ONE. 5 (10): e13543. Covey T M, Putta S, Cesano A. Single cell network profiling (SCNP): mapping drug and target interactions. Assay Drug Dev Technol. 2010;8:321-43.

I. Single Cell Network Profiling (SCNP)

Single cell network profiling (SCNP) is a method that can be used to analyze activatable elements, such as phosphorylation sites of proteins, in signaling pathways in single cells in response to modulation by signaling agonists or inhibitors (e.g., kinase inhibitors). Other examples of activatable elements include an acetylation site, a ubiquitination site, a methylation site, a hydroxylation site, a SUMOylation site, or a cleavage site. Activation of an activatable element can involve a change in cellular localization or conformation state of individual proteins, or change in ion levels, oxidation state, pH etc. It is useful to classify cells and to provide diagnosis or prognosis as well as other activities, such as drug screening or research, based on the cell classifications. SCNP is one method that can be used in conjunction with an analysis of cell health, but there are other methods that may benefit from this analysis. Embodiments of SCNP are shown in references cited herein. See for example, U.S. Pat. Nos. 7,695,924, 8,187,885, and 8273,544.

In one embodiment, SCNP can be used to generate a cell signaling profile. In another embodiment, SCNP can be used to measure apoptosis in cells stained with an antibody with specific affinity to cleaved PARP (cPARP), for example, after the cells have been exposed to one or more modulators, such as chemotherapy drugs or other treatments. Other cell health markers may be quantified as well. In one embodiment, the one or more cell health markers can be MCL-1 and/or cPARP. See for example, PCT/US2011/48332.

A significant fraction of cells with high cleaved PARP levels or low MCL-1 levels, before or without treatment with, e.g., a modulator, can indicate that some cells are undergoing apoptosis before treatment with a modulator. For some experiments, the activation state or activation level of an activatable element in an untreated sample of cells may be attributable to cells undergoing apoptosis due to one or more reasons related to sample processing (e.g., shipment conditions, cryogenic storage, thawing of cryogenically stored cells, etc.). If the apoptotic cells are not physically removed from the analysis, or data from apoptotic cells is not removed from an analysis of cell signaling data, apoptotic cells (which can be cleaved PARP positive or MCL-1 negative) can negatively impact the measurement of treatment (e.g., with a modulator) induced activation of an activatable element, e.g., phosphorylation of a phosphorylation site, and cause a misleading view of the signaling potential for the specific cell population being studied. See the references cited above, including the patents and applications, all of which are hereby incorporated by reference in their entireties.

II. Quality Control Methods

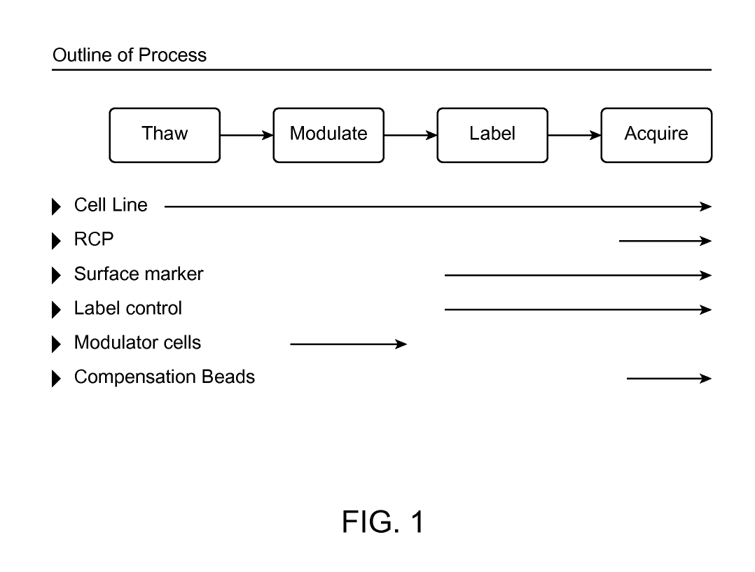

It is highly desirable to be consistent and to minimize errors with medical testing, including clinical testing, drug discovery, patient monitoring and prognostic or preclinical tests. These errors may affect patient life as well as jeopardizing the progress of a diagnostic test or a new drug. One embodiment of the present invention enables a researcher to monitor the fidelity of the assay under different variables, for example different operators, lots, reagents, cell lines, times, geographical locations, sample holders (such as tubes, wells or plates) and runs. One embodiment of the present invention is a method to provide control cells or beads or both for a plurality of phases of the assay. One or more control modules may be employed to monitor the process from start to finish. For example, one control module may span more than one step and others may span less steps. See FIG. 1 for example.

Previously filed patent applications have elements used in the present process and include the use of control beads, the use of monitoring software, computer systems and the use of automation (see U.S. Pat. No. 8,187,885 and U.S. Ser. Nos. 12/776,349, 12/501,274 and 12/606,869, respectively). All applications are hereby incorporated by reference in their entireties.

One embodiment of the present invention uses the SCNP process in which samples are thawed after cryopreservation, modulated, stained, and acquired. One embodiment of the present invention uses one or more of the SCNP process steps. For example, it is envisioned that the process may also use fresh samples which the thaw step is omitted. One embodiment of the present invention uses one or more of the SCNP process steps can have multiple sub-steps which can be monitored. For example, the labeling step may have multiple sub-steps such as more than binding element. See FIG. 1. Some control processes described herein will be useful for all process steps and others will be more focused on one or two steps. It is envisioned that the controlled process can include multiple sub-steps with a given process step.

FIG. 1 shows one embodiment of the sample treatment process broken down into four steps; Thaw, Modulation, Labeling, and Acquisition. See also U.S. Pat. No. 8,227,202 for an example of the general SCNP method. Also, several of the monitoring steps are shown by arrows for the steps that they monitor. For example, the use of cell lines is applied across the entire process, but the use of rainbow beads would be across the acquisition steps. If a variance is noticed in any monitoring embodiment, then other embodiments may be used to confirm the variance and its origin. For example, if a problem arises and several embodiments of the invention provide for showing what certain steps of the process were run properly, then other steps which were not determined to run properly can be the focus of the inquiry. Also, algorithms can be used after a variance is discovered to detect the root cause of the fault as they usually follow patterns that are automatically detectable.

One embodiment of the present invention includes the use of standard cells, such as cell lines with known genetic make-up, or banked cell aliquots from a healthy donor, in the assay as live, functional controls to monitor the all or part of the assay as it proceeds from start to finish along with the sample cells. For example, in one embodiment, an effective amount of the standard cells or healthy donor cells are placed in wells of a holder (such as a micro titer plate) along with the samples to be assayed. The standard cells or healthy donor cells are treated in the same fashion as the sample cells to be tested and therefore any variances with the data from the quality control reagents, such as the cells or beads, can be linked to the data for the samples. The sample data can then be excluded or adjusted/normalized based on the variances from preset ranges.

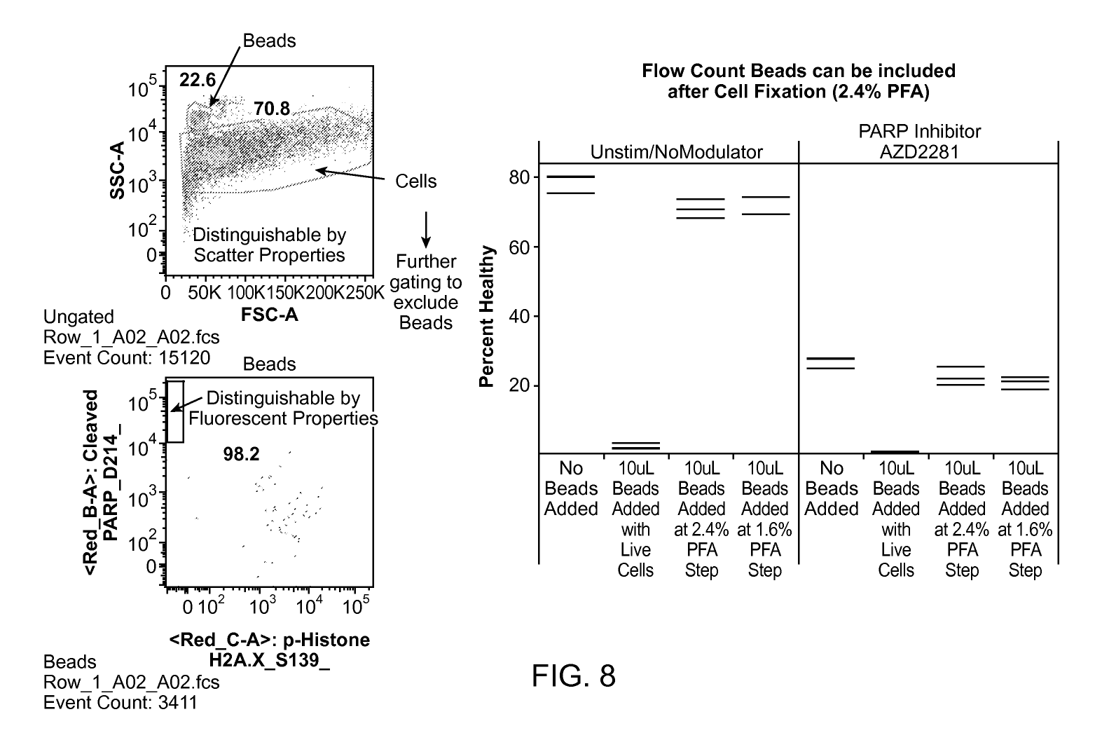

Another embodiment of the invention uses control beads, such as rainbow control particles (RCP) or Flow Count beads, in wells of a microtiter plate to monitor assay variability from sources, including but not limited to, instruments, plates and operators, for example. See U.S. Pat. No. 8,187,885 which is hereby incorporated by reference in its entirety. In one embodiment, certain beads are used in the labeling step (i.e. Ig Capture Beads) and other beads used in the acquisition step (i.e. Rainbow calibration particles) steps. Another embodiment of the invention, the beads are used in every well (i.e. Flow Count Beads, Beckman Coulter), in another embodiment, the beads are used in separate wells in the microplate. FIG. 8 shows an example of SCNP data collected with Flow Count beads in wells with sample cells.

Use of quality control reagents in the well with the test samples allows for direct measurement of the conditions relating to the samples. Use of quality control reagents in parallel processed wells is an indirect detection of the conditions relating to the samples.

Another embodiment of the invention is the use of label/stain controls to monitor the labeling process. Standardized cells may be obtained and used for this part of the process. In one embodiment, the cells may be preserved, such as by lyophilization, reconstituted and added to wells at the labeling and acquisition stages to ensure that the labeling step was working properly. Standard cells that can be used include the cell lines that are described herein. Lyophilized cells can be previously treated or stimulated, fixed, and lyophilized in bulk. These standard lyophilized cells are not suitable for the stimulation procedures because they are already treated and fixed. Lyophilized cells may be purchased from sources such as Becton Dickinson, San Jose, Calif. See for example, BD Phosphoflow T Cell Kit Lyophilized cells. See also U.S. Pat. No. 5,059,518. Another use of lyophilized staining controls would be to quality control (QC) and confirm the flow cytometer compensation values.

Another embodiment of the invention provides time records for each step of the process. An operator or automatic system would track each activity and the time of its start, stop and duration and then generate a report to the user in paper or electronic form. These records help facilitate identification of problems when they are due to activities that are time, not method based. An example would be if there was a variance that occurred during the morning start up, but not during a later running Time records would indicate when the error(s) occurred and this information would provide insight into the potential cause.

Another embodiment of the invention monitors the performance of phenotypic markers, such as cell surface or intracellular markers of the patient cell samples or cell standards, such as healthy donor sample cells or cell lines. The monitoring of the phenotypic markers performance can be the intensity of any signal received from the labeled antibodies designed to affix to the surface marker in a cell standard. Any typical phenotypic markers may be used and if values outside of an acceptable range or threshold are obtained in the monitoring step, then an alert may be manually or automatically raised to a user or in the report. Automatically raising the alert can allow a user to interrupt an ongoing experiment and to allow salvage of precious cells and reagents, depending on the fault. Surface markers are detailed below. Kits for measuring cell surface proteins include BD Lyoplate technology available from Becton Dickinson, San Jose, Calif. The addition of antibodies can also be measured using a kit for measuring antibodies in solution such as a mouse version of the Total Human IgG Flex Set produced by Becton Dickinson, San Jose, Calif. The kit is an application of cytometric bead array technology that measures human antibodies in plasma; a similar kit could be used to measure mouse antibodies specific to human surface marker proteins or intracellular proteins. The data would be used to monitor the addition of antibodies to the cells. A related product used for establishing an antibody's spectral emission, BD™ CompBead Plus available from Becton Dickinson, San Jose, Calif., could also be used to capture and measure antibodies in solution.

Suitable surface markers used to monitor the performance of phenotypic markers should be consistent across cells of the same donor which are split across multiple wells. Monitoring the surface markers of these cells should show a consistent narrow range of values. Cell surface markers should also be consistent across multiple patients in a predictable manner and range. Typical ranges for values for surface markers across the same patient should be between 1 and 5% or 1 and 10%. Values outside of this range may be acceptable or subject to normalization. Intracellular markers may also be used across cells of the same patient across multiple wells.

Another embodiment of the invention uses specific cell lines that predictably respond to different modulators to monitor the modulator step. This control is useful as a check on whether the modulation process step was working correctly when the patient sample was assayed. These cells can be introduced into the microtiter plate (microplate) in particular wells and then modulated along with the patient samples and cell lines. A monitoring test may be run on the microplate to ensure that the modulator is present. Also, the modulators can be stamped or aliquotted into another well or plate to determine if the concentration was correct. An ELISA or equivalent test (i.e. Cytometric Bead Array) may be used to make this determination. Another embodiment of the invention includes the use of beads measuring protein modulators such as cytokine bead arrays, which would ensure that the appropriate modulators, at the appropriate concentration are being used. This embodiment would avoid the need to have live cell controls which respond to the specific modulators of interest.

Automation and informatics on the automated process steps are useful in some embodiments of the invention. For example, they are useful to acquire data on each of the monitoring steps described herein and then to identify any out of variance values to an operator for remedial action. The operator can stop a run, abbreviate a run, or implement a corrective action during the course of a run/study to save precious resources. Computational informatics approaches envisioned to be used with the invention, include but are not limited to, density estimation. Peak finding can be used to automatically gate the events in any well to identify cells of interest. In one example, auto-gating uses the distribution CD34 expression is to differentiate between the cell lines GDM-1 and RS;411 that are assayed as a mixture in a single well. Several metrics of interest like median fluorescence intensity (MFI), or those that compare protein expression levels between modulated and unmodulated levels (for example: Fold change, Mann-Whitney U metric) can be computed. Appropriate ranges for each control step are dependent on each step, but are within 1 and 15% for these metrics, or less than 10% or within 2 and 10%. In certain embodiments, manual or automated gating may also be used with informatics on the automated process steps applied after the manual steps are performed.

One embodiment of the invention places the output data from each and any of the monitoring methods described herein into a database. This database provides a large volume of data to measure the results of future monitoring assays and to provide a context for those future results. Data can be gathered through each stage for each monitoring process. In one embodiment, there are greater than 25, 50, 100, 500, or 1,000 records for each/any monitoring step in the invention. Output data from new runs can be compared to the database and an operator can be alerted on a report if variation over a particular threshold is noted in the database. The threshold can be set at 5%, 10%, 15%, or 20%.

In one embodiment the monitoring of surface markers will occur at the last two stages of the process, Labeling and Acquisition. (Labeling can also be referred to as “staining”). Factors that may create a variance in the labeling and acquisition steps include cell permeablization (there are many reagents, but methanol is typical), labeling, reagents, cell fixation, among other causes Variances indicate that either of these two steps had a deviation from the norm. This monitoring step is also used to corroborate the cell line monitoring as both are used in the last two steps. Other steps that are used to confirm the last two steps include the use of the rainbow beads and the cell lyophilization controls.

One embodiment of the present invention establishes consistency between multiple operators, plates and assay runs in a multiparametric assay. In one embodiment, the assay uses live cells in a functional test. In another embodiment, the assay uses the live cells in a functional, single cell analysis, such as that described above for SCNP. It is desirable to test samples and monitor the accuracy, consistency or fidelity of the individual steps in the process. Early detection of any problems allows early correction.

In one embodiment of the invention, samples are arrayed in wells for testing. A microtiter plate may be used as one example of a holder; other formats for sample assay are acceptable as described herein. One embodiment uses standard cells in the same or another microtiter plate to test along with the sample cells to determine if the SCNP process is working properly. Standard cells can be an appropriate number of cells from standard cell lines with known genetic make-up or other standard cells that are deemed to behave similarly. The performance of a certain numbers of cells can be measured at the start of a test or other points in the assay, such as final acquisition or final analysis. Typical numbers of cells may be between 1,000 and 10,000,000 cells, or between 50,000 and 5,000,000 cells, or between 75,000 and 500,000. In one embodiment, the output from the quality control test will be to measure the similarity of responses for specific measurements in SCNP tested over batches of the standard cells having a similar number of cells. In one embodiment, the cells are run along with the sample cells in the same microtiter plate. In one embodiment, the standard cells are in the same well as the sample cells in the cell microtiter plate.

In another embodiment, rainbow calibration particles (RCP) are added to the microtiter plate to monitor signal detection consistency between instruments, such as flow cytometers, arrays, and individual microtiter plates. See U.S. Pat. No. 8,187,885 which is hereby incorporated by reference in its entirety for all purposes. In one embodiment, RCP are added to a row of wells in a microtiter plate and the calibrations are performed plate by plate. In one embodiment, 66 parameters may be collected—8 labeled each having different fluorescent labels at 8 (given wavelengths of electromagnetic radiation) intensities, and 2 scatter properties (size and granulation). Peaks numbered by intensity from low (Peak 1) to high (Peak 8). Peak 1 is below instrument noise level, is always excluded from analysis. Peak 2 may show the highest variance as it has the next lowest noise level. In is also envisioned that his assay could be used with mass cytometry. Mass cytometry, uses lanthanide isotopes are attached to antibodies to overcome the fluorescent labeling limit proved by spectral overlap. This multiplexed method has been demonstrated to allow for 30 different labels to be used in a given cytometry assay. This mass cytometry could theoretically allow the use of 40 to 60 distinguishable labels. See Tanner et al. Spectrochimica Acta Part B: Atomic Spectroscopy, 2007 March;62(3):188-195. See also, U.S. Patent Publications 2012/0056086, 2011/0253888, 2009/0134326, and 2011/0024615 which are incorporated by reference in their entireties.

In one embodiment, a flow cytometer is used in the assay and multiple characteristics may be measured. In one embodiment, surface markers, intracellular markers, or other characteristics may be measured. In one embodiment, between 1 and 8 channels are measured for a standard flow cytometer described below. In another embodiment between 1 and 75 channels are measured with other cytometers, such as a CyTOF (mass cytometer/spectrometer) also described below.

In one embodiment, cell lines are used as the standard cells to monitor the course of the whole or part of the assay. In one embodiment of the invention some exemplary cell lines are those that match with the type of cell samples being tested. For example, if a diagnostic for AML is being run, then AML cells lines can be used. Other cell lines may also be used as well. Non-limiting exemplary cell lines that can be used with the assay, include: GDM-1, RS;411, U-937, Jurkat, Ramos, HeLa, DU-145, LNCaP, MCF-1, MDA-MB-438, PC-3, T47D, THP-1, U-87, SH-SY5Y, Saos-2, BaF/3, 293T/17 and others. Cell lines may be obtained from commercial depositories such as those published by WIPO recognized under the Budapest Treaty, for example, ATCC (Manassas, Va.), Advanced Biotechnology Center (Genoa, Italy), Agricultural Research Service (Peoria, Ill.), and other sources such as the NCI and Coriell, Camden N.J.

In one embodiment, it is important to determine that the process is working without error and that the cell line data is consistent as a measure of error free operation. In one embodiment, greater than 85%, 90%, or 95% of the results from the analysis of the standards have coefficients of variation (CV) within 20%, 15%, 10%, 7%, or 5%. In some embodiments the values that are measured are the fluorescent intensity values for the labels measured by a detection instrument, such as a flow cytometer, although other values may be measured. In some embodiments the values are metrics based on relative intensity, calibrated intensity and calculated metrics in the QC process, also included are the mean or median intensity such as Log2Fold values (see U.S. Ser. Nos. 13/083,156 and 13/566,991for other metrics). In some embodiments the values are distribution changes based on the Mann-Whitney U statistic.

Particular embodiments of high throughput flow cytometry system may utilize microtiter type plates. The plates may be conventional and commercially available, or they may be a custom design. The number of wells may be 96, 384, 1536 or other standard sizes. The volume may be as stated above, at least 1, 2, 3, 4, 5, 6, or 7 or more microliters. Microtiter plates may be obtained from commercial suppliers such as Becton Dickinson or Beckman Coulter. In a particular embodiment, the microtiter plate may have predeposited reagents. Other holders may be used as described herein.

In some embodiments, the cell surface markers that can be monitored for stain controls or sample surface markers include some or all, but are not limited to, CD3, CD4, CD5, CD7, CD8, CD11b, CD11c, CD14, CD15, CD16, CD19, CD20, CD22, CD25, CD27, CD28, CD33, CD34, CD38, CD40, CD45, CD56, CD69, CD71, CD80, CD117, CD138, CD161, CD235a, CD235b, Ter119, GP-130, IgM, IgD, IgE, IgG, IgA, CCR5, CCR3, TLR2, or TLR4.

CD3, also known as T3, is a member of the immunoglobulin (Ig) superfamily that plays a role in antigen recognition, signal transduction and T cell activation. It is found on all mature T lymphocytes, NK-T cells, and some thymocytes. CD4 is also a member of the Ig superfamily, which participates in cell-cell interactions, thymic differentiation, and signal transduction. It is primarily expressed on most thymocytes, a subset of T cell and monocytes/macrophages. CD7 is found on T cells, NK cells, thymocytes, hematopoietic progenitors and monocytes. CD7 is also expressed on ALL and some AML cells. CD11b is a member of the integrin family, primarily expressed on granulocytes, monocytes/macrophages, dendritic cells, NK cells, and subsets of T and B cells. CD14 is a GPI-linked membrane glycoprotein, also known as LPS receptor. It is expressed at high levels on macrophages, monocytes and at low level on granulocytes. CD33 is a sialoadhesion Ig superfamily member expressed on myeloid progenitors, monocytes, granulocytes, dendritic cells and mast cells. It is absent on normal platelets, lymphocytes, erythrocytes and hematopoietic stem cells. CD34 is a type I monomeric sialomucin-like glycophosphoprotein. It is selectively expressed on the majority of hematopoietic stem/progenitor cells, bone marrow stromal cells, capillary endothelial cells, embryonic fibroblasts, and some nervous tissues. It is commonly used marker for identifying human hematopoietic stem/progenitor cells. CD45 is commonly known as the leukocyte common antigen. It is a transmembrane tyrosine phosphatase expressed on all hematopoietic cells, except erythrocytes and platelets. It is a signaling molecule that regulates a variety of cellular processes including cell growth, differentiation, cell cycle, and oncogenic transformation. It plays a critical role in T and B cell antigen receptor-mediated activation. CD71 is a type II heterodimeric transmembrane glycoprotein also known as the transferrin receptor. It is expressed on proliferating cells, reticulocytes, and erythroid precursors. CD71 plays a role in the control of cellular proliferation by facilitating the uptake of iron via ferrotransferrin binding and the recycling of apotransferrin to the cell surface. CD235a is also known as glycophorin A and CD235b is also known as glycophorin B, major sialoglycoproteins expressed on the red blood cell membrane and erythroid precursors. Mature, non-nucleated red blood cells are characteristically CD235a and/or CD235b positive, but CD45 and CD71 negative.

In another embodiment intracellular phenotypic markers of cell lineage can be monitored for some or all stain controls such as TLR3, TLR7, TLR8, TLR9, PTEN, GAPDH, Actin, Tubulin as well as other markers.

In another embodiment includes algorithmic methods to determine the causes of faults during the process which can be automated. For example, certain root causes can display with a particular fault pattern. This embodiment can involve a rules based system for seeing if a pattern or signature exists when a fault arises. For example, if all surface markers are defective, it could suggest that there is a problem with the fixative. Testing of an automated system can be performed by intentionally causing a fault and observing the downstream effects of the fault. Creating multiple individual or combined faults in different areas of the process can create a database of results that can be employed for diagnostics later during the actual running of the process. In another embodiment, spare plates may be employed to run the process, without the addition of the test sample cells, to check if certain reagents, like the modulators, are dispensed properly by the automated fluidic system. In one example, the weight of the plate can be measured by the system to determine if the automated fluidic system dispensed the proper amount. In another example, cytometric bead arrays can be employed to determine if sufficient amounts of modulators were added to the individual wells of a microtiter plate. In another embodiment, different distinguishable beads can be mixed with the modulators to identify any problems with that step. In another embodiment, beads can be differentially labeled and inserted into different stain cocktails for a similar purposeIn another embodiment, automatic gating can be used as applied to cell line monitoring to clean up the signal and for surface marker or intercellular signaling monitoring, to narrow the range of cells under review. For example, when using cell lines to monitor the process as described below, we assume that the signals arise from healthy cells. However, there may be between 10-20% of “debris” which can complicate the monitoring process. An automated gating program can be employed to remove “debris” and to focus on the narrower range of signals received from the live cells from the cell lines. Additionally, the present process monitors cell surface markers and intercellular signaling monitoring. Automated gating can be used to focus the analysis only on cell surface markers or intercellular signaling readouts that are combined with another characteristic, such as a specific cell type, like T cells. In one example, auto-gating uses the distribution CD34 expression is to differentiate between the cell lines GDM-1 and RS;411 that are assayed as a mixture in a single well. So that the analysis can be focused on the presence of CD3 on T cells, for example. In another example auto-gating may be used to enable monitoring intracellular signaling, For example, auto-gating could allow for signaling in specific cell types to be studies such as T cell, B cells, monocytes or other relevant cell populations may be assessed.

In one embodiment of the invention a quality control process uses the method and kits described in U.S. Pat. No. 8,187,885 which is hereby incorporated by reference in its entirety. The beads can be used as a process control to monitor well to well shifts and as a tool to normalize the signal between wells and or plates. They can be used in a flow cytometer with fluorescent labels or mass spectrometers with metal labels (for example, lanthanides as shown below). The beads may be incorporated into the same holders for/with the cells or along with the cells in separate holders. Holders are described herein. In one embodiment is a microtiter plate. When a microtiter plate is used, beads can be placed in a row of separate wells along with the sample cells and standard cells as an example.

One embodiment of the present invention involves the use of the preset ranges of acceptable values allow for exclusion of test data from samples or the basis for normalizing test data based on the deviation from the preset range of acceptable values. Normalization methods are known to those of skill in the art. For example, normalization refers to the creation of shifted and scaled versions of statistics, where the intention is that these normalized values allow the comparison of corresponding normalized values for different datasets in a way that eliminates the effects of certain gross influences, as in an anomaly time series. Some types of normalization involve only a rescaling, to arrive at values relative to some size variable. In an experimental context, normalizations are used to standardize microarray data to enable differentiation between real (biological) variations in gene expression levels and variations due to the measurement process. In microarray analysis, normalization refers to the process of identifying and removing the systematic effects, and bringing the data from different microarrays onto a common scale. The same usage may be applied in the present invention.

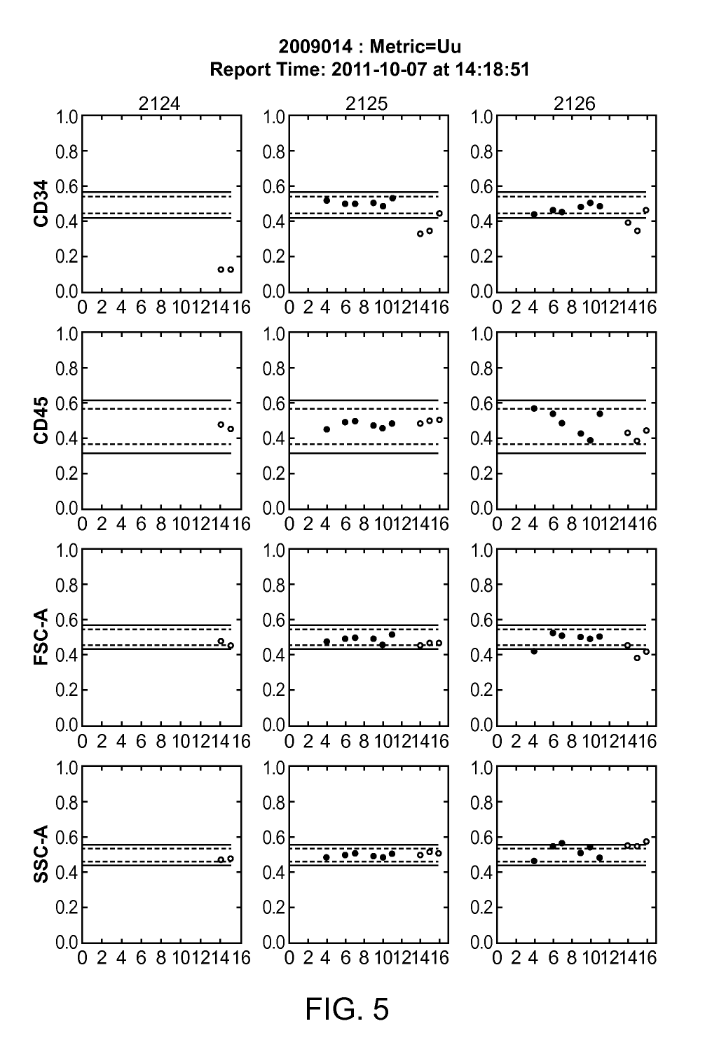

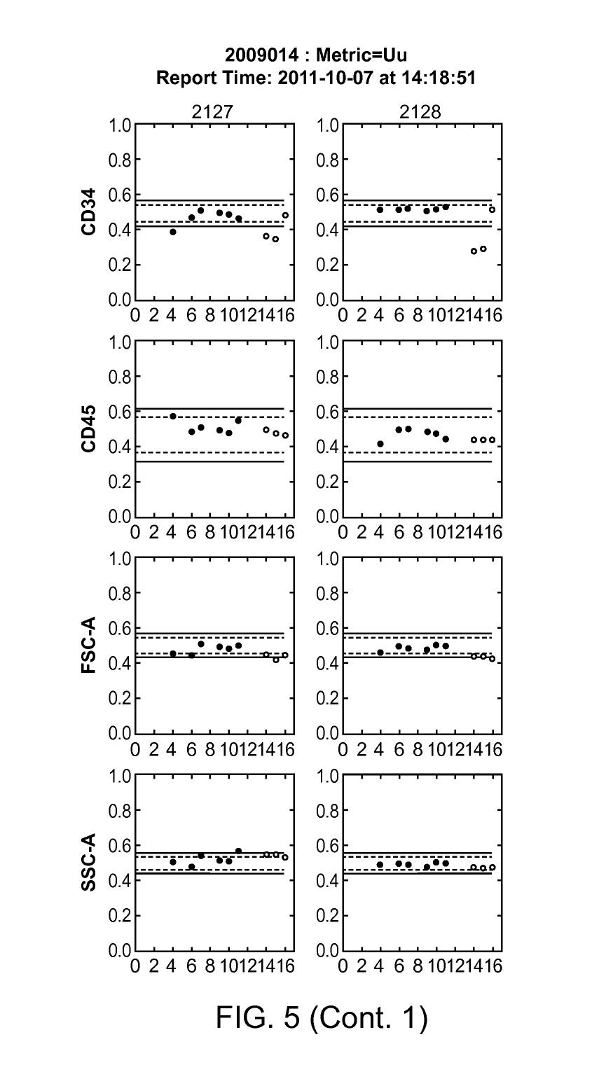

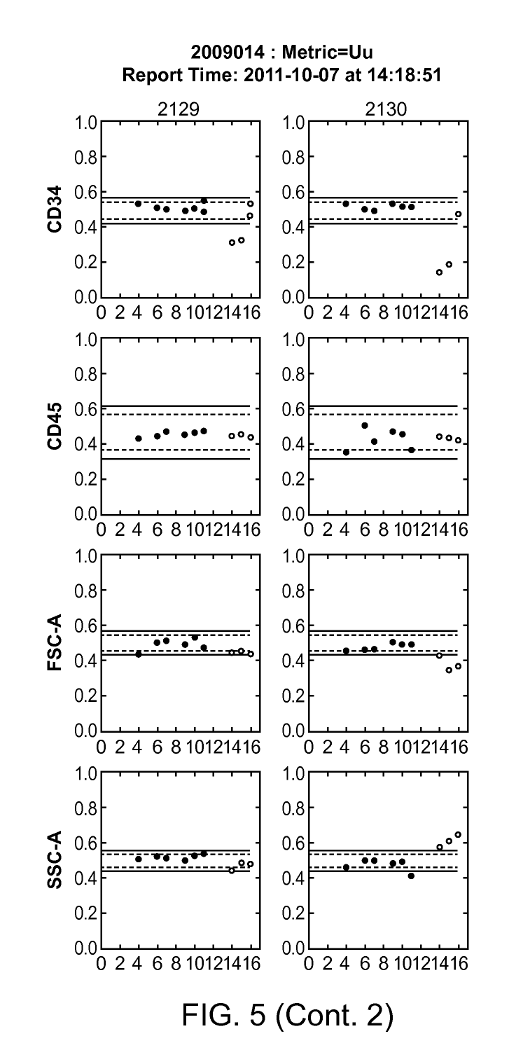

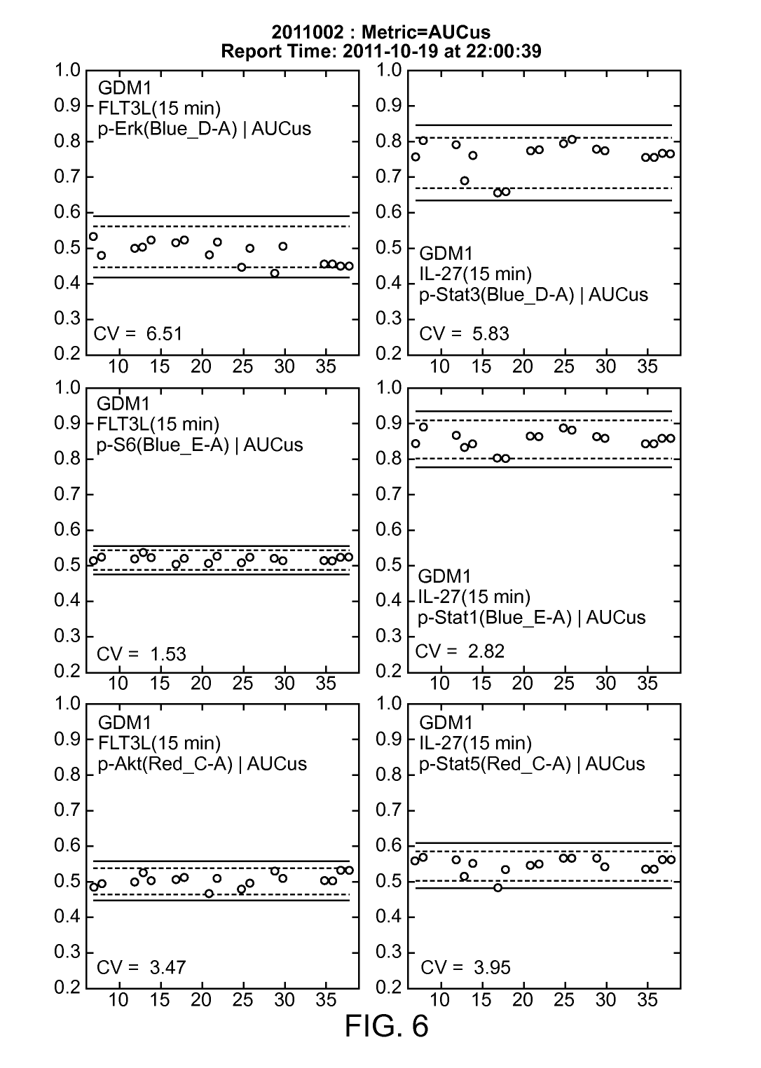

Preset ranges can be used as boundaries for acceptable values and they depend on the QC measurement to be made. For example, performance of any of the QC methods recited herein can be designated as acceptable if within a range of values. For example, see FIGS. 5, 6, and 7 which show bars for the placement of the actual results of the present tests. One set of narrowly placed bars may indicate one level of acceptance and more widely spaced bars can indicate a broader level of acceptance. Deviations of 1%, 5%, 10%, 15%, 20% or more may be acceptable with or without exclusion or correction by normalization or other methods. Data that is outside of the range or bars can indicate an error in the processing of the QC standard and therefore the test data is erroneous. Exclusion or correction by normalization will be performed.

Preset ranges may be based on many of the measurement metrics present in U.S. Ser. No. 13/566,991, or those shown the FIG. 5, 6, or 7. Example, metrics for the ranges may include MFI, AUC, Uᵤ, FSC, SSC, or other criteria that are preferably accurately and consistently measured.

One embodiment of the present invention includes a calibration kit which comprises several populations of fluorescent microbeads, at least one population being surface-dyed microbeads containing one or more non-overlapping fluorescent dyes, and at least two populations being internally-dyed microbeads with different amounts of one or more non-overlapping fluorescent dyes. By the terms “microbead”, “bead”, or “particle” as used herein is meant any solid particle, of virtually any shape, suitable for measurement by a fluorescence instrument. By “fluorescent dye” as used herein is meant any dye, molecule, complex, or particle that may be excited by a given wavelength of electromagnetic radiation, and emit photons of another wavelength. It should be understood that the terms “fluorescent dye”, “fluorescent probe”, “fluorescent molecule”, “fluorophore”, “fluorochrome”, “fluorescent nanocrystal”, and grammatical equivalents thereof, may be used interchangeably herein to refer to a fluorescent dye. By “overlapping” as used herein is meant when excited by any given wavelength of light, a first fluorescent dye emits some photons with the same wavelength as those emitted by a second fluorescent dye. Microbeads containing multiple overlapping fluorescent dyes have different fluorescence excitation and emission spectra depending on the number of molecules of dye per bead, as illustrated by Wang et al., Cytometry (2008). Consequently, the disclosed invention comprises calibration microbeads with one or more non-overlapping fluorescent dyes, so the excitation and emission spectra are comparable between bright and dim microbeads, and therefore the slope and linearity of the detector can be accurately determined with or without compensation algorithms depending on the fluorescent labels used.

Many types of beads and dyes (and other variables) can be used. Examples are shown in U.S. Pat. No.8,187,885. In some embodiments of the present invention, microbeads may be obtained from commercial suppliers, including: Bangs Laboratories, Inc, 9025 Technology Drive, Fishers, Ind. 46038-2886; Life Technologies Corporation, 5791 Van Allen Way, Carlsbad, Calif. 92008; Brookhaven Instruments Limited, Chapel House, Stock Wood Redditch, Worcestershire B96 6ST, UK; Spherotech, Inc., 27845 Irma Lee Circle, Unit 101, Lake Forest, Ill. 60045; Polysciences, Inc., 400 Valley Road, Warrington, Pa. 18976; BD, 1 Becton Drive, Franklin Lakes, N.J., 07417; Beckman Coulter, Brea, Calif. (now Danaher).

Fluorophores can be either “small molecule” fluors, or proteinaceous fluors (e.g. green fluorescent proteins and all variants thereof). Suitable fluorophores include, but are not limited to, 1,1′-diethyl-2,2′-cyanine iodide, 1,2-diphenylacetylene, 1,4-diphenylbutadiene, 1,6-Diphenylhexatriene, 2-Methylbenzoxazole, 2,5-Diphenyloxazole (PPO), 4-(dicyanomethylene)-2-methyl-6-(p-dimethylaminostyryl)-4H-pyran (DCM), 4-Dimethylamino-4′-nitrostilbene, 4′,6-Diamidino-2-phenylindole (DAPI), 5-ROX, 7-AAD, 7-Benzylamino-4-nitrobenz-2-oxa-1,3-diazole, 7-Methoxycoumarin-4-acetic acid, 9,10-Bis(phenylethynyl)anthracene, 9,10-Diphenylanthracene, Acridine Orange, Acridine yellow, Adenine, Allophycocyanin (APC), AMCA, AmCyan, Anthracene, Anthraquinone, APC, Auramine O, Azobenzene, Benzene, Benzoquinone, Beta-carotene, Bilirubin, Biphenyl, BO-PRO-1, BOBO-1, BODIPY FL, Calcium Green-1, Cascade Blue™, Cascade Yellow™, Chlorophyll a, Chlorophyll b, Chromomycin, Coumarin, Coumarin 1, Coumarin 30, Coumarin 314, Coumarin 343, Coumarin 6, Cresyl violet perchlorate, Cryptocyanine, Crystal violet, Cy2, Cy3, Cy3.5, Cy5, Cy5.5, Cy7, Cytosine, DA, Dansyl glycine, DAPI, DiI, DiO, DiOCn, Diprotonated-tetraphenylporphyrin, DsRed, EDANS, Eosin, Erythrosin, Ethidium Monoazide, Ethyl p-dimethylaminobenzoate, FAM, Ferrocene, FI, Fluo-3, Fluo-4, Fluorescein, Fluorescein isothiocyanate (FITC), Fura-2, Guanine, HcRed, Hematin, Histidine, bhy67, Hoechst 33258, Hoechst 33342, IAEDANS, Indo-1, Indocarbocyanine (C3)dye, Indodicarbocyanine (C5)dye, Indotricarbocyanine (C7)dye, LC Red 640, LC Red 705, Lucifer yellow, LysoSensor Yellow/Blue, Magnesium octaethylporphyrin, Magnesium octaethylporphyrin (MgOEP), Magnesium phthalocyanine (MgPc), Magnesium tetramesitylporphyrin (MgTMP), Magnesium tetraphenylporphyrin (MgTPP), Malachite green, Marina Blue®, Merocyanine 540, Methyl-coumarin, MitoTracker Red, N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, Naphthalene, Nile Blue, Nile Red, Octaethylporphyrin, Oregon green, Oxacarbocyanine (C3)dye, Oxadicarbocyanine (C5)dye, Oxatricarbocyanine (C7)dye, Oxazine 1, Oxazine 170, p-Quaterphenyl, p-Terphenyl, Pacific Blue®, Peridinin chlorophyll protein complex (PerCP), Perylene, Phenol, Phenylalanine, Phthalocyanine (Pc), Pinacyanol iodide, Piroxicam, POPOP, Porphin, Proflavin, Propidium iodide, Pyrene, Pyronin Y, Pyrrole, Quinine sulfate, R-Phycoerythrin (PE), Rhodamine, Rhodamine 123, Rhodamine 6G, Riboflavin, Rose bengal, SNARF®, Squarylium dye III, Stains-all, Stilbene, Sulforhodamine 101, SYTOX Blue, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), tetramethylrhodamine, Tetraphenylporphyrin (TPP), Texas Red® (TR), Thiacarbocyanine (C3)dye, Thiadicarbocyanine (C5)dye, Thiatricarbocyanine (C7)dye, Thiazole Orange, Thymine, TO-PRO®-3, Toluene, TOTO-3, TR, Tris(2,2′-bipyridyl)ruthenium(II), TRITC, TRP, Tryptophan, Tyrosine, Uracil, Vitamin B12, YO-PRO-1, YOYO-1, Zinc octaethylporphyrin (ZnOEP), Zinc phthalocyanine (ZnPc), Zinc tetramesitylporphyrin (ZnTMP), Zinc tetramesitylporphyrin radical cation, and Zinc tetraphenylporphyrin (ZnTPP). Suitable optical dyes are described in the 1996 Molecular Probes Handbook by Richard P. Haugland, hereby expressly incorporated by reference.

In some embodiments, the fluorescent dye may be an Alexa Fluor® dye, including Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750 (Life Technologies Corporation (formerly Invitrogen), 5791 Van Allen Way, Carlsbad, Calif. 92008).

In some embodiments, the fluorescent dye may be a tandem fluorophore conjugate, including Cy5-PE, Cy5.5-PE, Cy7-PE, Cy5.5-APC, Cy7-APC, Cy5.5-PerCP, Alexa Fluor® 610-PE, Alexa Fluor® 700-APC, and Texas Red-PE. Tandem conjugates are less stable than monomeric fluorophores, so comparing a detection reagent labeled with a tandem conjugate to reference solutions may yield MESF calibration constants with less precision than if a monomeric fluorophore had been used.

In some embodiments, the fluorescent dye may be a fluorescent protein such as green fluorescent protein (GFP; Chalfie, et al., Science 263(5148):802-805 (Feb. 11, 1994); and EGFP; Clontech--Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), cyan fluorescent protein (CFP), and enhanced yellow fluorescent protein (EYFP; 1. Clontech Laboratories, Inc., 1020 East Meadow Circle, Palo Alto, Calif. 94303). In some embodiments, the fluorescent dye is dTomato, FlAsH, mBanana, mCherry, mHoneydew, mOrange, mPlum, mStrawberry, mTangerine, ReAsH, Sapphire, mKO, mCitrine, Cerulean, Ypet, tdTomato, Emerald, or T-Sapphire (Shaner et al., Nature Methods, 2(12):905-9.(2005)). All of the above-cited references are expressly incorporated herein by reference.

In some embodiments, the fluorescent dye may be a fluorescent semiconductor nanocrystal particle, or quantum dot, including Qdot® 525 nanocrystals, Qdot® 565 nanocrystals, Qdot® 585 nanocrystals, Qdot® 605 nanocrystals, Qdot® 655 nanocrystals, Qdot® 705 nanocrystals, Qdot® 800 nanocrystals (Life Technologies Corporation (formerly Invitrogen), 5791 Van Allen Way, Carlsbad, Calif. 92008). In some embodiments, the fluorescent dye may be an upconversion nanocrystal, as described in Wang et al., Chem. Soc. Rev., 38:976-989 (2009), which is hereby incorporated by reference in its entirety.

In one embodiment of the invention the methods and reagents shown in U.S. Pat. No. 8,187,885 are employed to monitor the performance of the assay. The beads can be added to different wells of a microtiter plate or other cell holder. The beads can also be added to the wells having the cell samples and standard cells.

To permit the use of a large number of labels, beads labeled with various isotopes can also be used with mass cytometer in the present process.

Activatable Elements

The methods and compositions described herein may be employed to examine and profile the status or activation level of any activatable element in a cellular pathway, or collections of such activatable elements. Single or multiple distinct pathways can be profiled (e.g., sequentially or simultaneously), or subsets of activatable elements within a single pathway or across multiple pathways can be examined (e.g., sequentially or simultaneously).

In some embodiments, apoptosis, signaling, cell cycle and/or DNA damage pathways are characterized in order to classify, profile, diagnosis, prognosis or predict drug response in one or more cells in an individual. The characterization of multiple pathways can reveal operative pathways in a condition that can then be used to classify one or more cells in an individual. In some embodiments, the classification includes classifying the cell as a cell that is correlated with a clinical outcome. The clinical outcome can be the prognosis and/or diagnosis of a condition, and/or staging or grading of a condition. In some embodiments, the classifying of the cell includes classifying the cell as a cell that is correlated with a patient response to a treatment. In some embodiments, the classifying of the cell includes classifying the cell as a cell that is correlated with minimal residual disease or emerging resistance. In some embodiments, the cell classification includes correlating a response to a potential drug treatment. In another embodiment, the present invention includes a method for drug screening. See also U.S. Pat. No. 8,227,202 and U.S. Ser. Nos. 12/432,720 and 61/048,886 for activatable elements.

As will be appreciated by those in the art, a wide variety of activation events can find use in the methods described herein. In general, activation can result in a change in the activatable protein that is detectable by some indication (termed an “activation state indicator”), e.g., by altered binding of a labeled binding element or by changes in detectable biological activities (e.g., the activated state has an enzymatic activity which can be measured and compared to a lack of activity in the non-activated state). Using one or more detectable events or moieties, two or more activation states (e.g., “off” and “on”) can be differentiated.

The activation state of an individual activatable element can be in the on or off state. As an illustrative example, and without intending to be limited to any theory, an individual phosphorylation site on a protein can activate or deactivate the protein. Phosphorylation of an adapter protein can promote its interaction with other components/proteins of distinct cellular signaling pathways. In another embodiment, the difference in enzymatic activity in a protein can reflect a different activation state. The terms “on” and “off,” when applied to an activatable element that is a part of a cellular constituent, can be used here to describe the state of the activatable element, and not the overall state of the cellular constituent of which it is a part.

The activation state of an individual activatable element can be represented as continuous numeric values representing a quantity of the activatable element or can be discretized into categorical variables. For instance, the activation state may be discretized into a binary value indicating that the activatable element is either in the on or off state. As an illustrative example, and without intending to be limited to any theory, an individual phosphorylatable site on a protein will either be phosphorylated and then be in the “on” state or it will not be phosphorylated and hence, it will be in the “off” state. See Blume-Jensen and Hunter, Nature, vol 411, 17 May 2001, p 355-365.

Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element can be measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell can be the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations. For more information on the measurement of activatable elements, specific activatable elements, signaling pathways, and drug transporters, see U.S. Pat. No. 8,227,202 and U.S. Ser. No. 12/910,769 or U.S. Pub. No. 2009/0269773, which are hereby incorporated by reference in their entireties.

In some embodiments, the activation levels of one or more activatable elements of a cell from a first population of cells and the activation levels of one or more activatable elements of a cell from a second population of cells are correlated with a condition. In some embodiments, the first and second homogeneous populations of cells are hematopoietic cell populations. In some embodiments, the activation levels of one or more activatable elements of a cell from a first population of hematopoietic cells and the activation levels of one or more activatable elements of cell from a second population of hematopoietic cells are correlated with a neoplastic, autoimmune or hematopoietic condition as described herein. Examples of different populations of hematopoietic cells include, but are not limited to, pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change can occur to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. The term “plurality” as used herein refers to two or more. In some embodiments, the activation level of at least about 2, 3, 4, 5, 6, 7, 8, 9, 10, or more than 10 intracellular activatable elements are determined. The term “plurality” as used herein refers to two or more. In other embodiments, the activation level of at least about 2, 3, 4, 5, 6, 7, 8, 9, 10, or more than 10 surface and intracellular activatable elements are determined. In some embodiments, the activation level of at least about 20, 30, 40, 50, or 60 surface and intracellular activatable elements are determined.

Activation states of activatable elements can may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In some embodiments, the activatable element is a protein. Examples of proteins that can include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 2006/0073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 12/432,720, 12/229,476 and Shulz et al, Current Protocols in Immunology 2007, 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1,Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPB, XIAP, Smac, Cdk4, Cdk 6, Cdk 2, Cdk1, Cdk 7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, STAT1, STAT3, STAT4, STAT5, STATE, p53, Ets-1, Ets-2, SPDEF, GABPα, Tel, Te12, WT-1, HMGA, pS6, 4EPB-1, eIF4E-binding protein, RNA polymerase, initiation factors, elongation factors.

In some embodiments, the methods described herein are employed to determine the activation level of an activatable element, e.g., in a cellular pathway. Methods and compositions are provided for the determination of a cell signaling profile (e.g., activation level of an activatable element) of a cell according to the activation level of an activatable element in a cellular pathway. Methods and compositions are provided for the determination of the cell signaling profile of a cell in a first cell population and a cell in a second cell population according to the activation level of an activatable element in a cellular pathway in each cell. The cells can be a hematopoietic cell, disease cell, immune cell, healthily/normal cells or standard control cell.

In some embodiments, the determination of the cell signaling profile of cells in different populations according to activation level of an activatable element, e.g., in a cellular pathway comprises classifying at least one of the cells, based on their cell signaling profile as a cell that is correlated with a clinical outcome. Examples of clinical outcomes, staging, as well as patient responses correlated with cell signaling profiles are shown above.

Signaling Pathways

In some embodiments, the methods described herein are employed to determine the activation level of an activatable element in a signaling pathway. In some embodiments, the cell signaling profile of a cell is determined, as described herein, according to the activation level of one or more activatable elements in one or more signaling pathways. Signaling pathways and their members have been extensively described. See (Hunter T. Cell Jan. 7, 2000;100(1): 13-27; Weinberg, 2007; and Blume-Jensen and Hunter, Nature, vol 411, 17 May 2001, p 355-365 cited above). Exemplary signaling pathways include the following pathways and their members: the JAK-STAT pathway including JAKs, STATs 2,3 4 and 5, the FLT3L signaling pathway, the MAP kinase pathway including Ras, Raf, MEK, ERK and Elk; the PI3K/Akt pathway including PI-3-kinase, PDK1, Akt and Bad; the NF-κB pathway including IKKs, IkB and NF-κB and the Wnt pathway including frizzled receptors, β-catenin, APC and other co-factors and TCF (see Cell Signaling Technology, Inc. 2002 Catalog pages 231-279 and Hunter T., supra.). In some embodiments, the correlated activatable elements being assayed (or the signaling proteins being examined) are members of the MAP kinase, Akt, NFkB, WNT, STAT and/or PKC signaling pathways. See the description of signaling pathways in U.S. Ser. No. 12/910,769 which is incorporated by reference in its entirety.

In some embodiments, methods are employed to determine the activation level of a signaling protein in a signaling pathway known in the art including those described herein. Exemplary types of signaling proteins include, but are not limited to, kinases, kinase substrates (i.e., phosphorylated substrates), phosphatases, phosphatase substrates, binding proteins (such as 14-3-3), receptor ligands and receptors (cell surface receptor tyrosine kinases and nuclear receptors). Kinases and protein binding domains, for example, have been well described (see, e.g., Cell Signaling Technology, Inc., 2002 Catalogue “The Human Protein Kinases” and “Protein Interaction Domains” pgs. 254-279).

Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGF-β receptors, BMP receptors, MEKKs, ASK, MLKs, DLK, PAKs, Mek 1, Mek 2, MKK3/6, MKK4/7, ASK1,Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKs, Pim1, Pim2, Pim3, IKKs, Cdks, Jnks, Erks, Erk1, Erk2, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/ DNA binding proteins, Ets, Elk, SMADs, Rel-A (p65-NFKB), CREB, NFAT, ATF-2, AFT, Myc, Fos, Spl, Egr-1, T-bet, HIFs, FOXOs, E2Fs, SRFs, TCFs, Egr-1, β-catenin, FOXOs, STAT1, STAT3, STAT4, STAT5, STAT6, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCyi, PLCy 2, STAT1, STAT3, STAT4, STAT5, STAT6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, pl4Arf, p27KIP, p21CIP, Cdk4, Cdk6, Cdk7, Cdk1, Cdk2, Cdk9, Cdc25,A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPB, Smac, Fodrin, Actin, Src, Lyn, Fyn, Lck, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, β-catenin, CrkL, GSK3α, GSK3β, and FOXO.

In some embodiments, the methods described herein are employed to determine the activation level of an activatable element in a signaling pathway. See also U.S. Ser. Nos. 12/432,720, 12/229,476 which are incorporated by reference in their entireties. Methods and compositions are provided for the determination of a cell signaling profile of a cell according to the status of an activatable element in a signaling pathway. Methods and compositions are provided for the determination of a cell signaling profile of cells in different populations of cells according to the status of an activatable element in a signaling pathway. The cells can be a hematopoietic cells. Examples of hematopoietic cells are shown above. In some embodiments, the determination of a cell signaling profile of cells in different populations of cells according to the activation level of an activatable element in a signaling pathway comprises classifying the cell populations as cells that are correlated with a clinical outcome. Examples of clinical outcome, staging, patient responses and classifications are shown above.

Binding Element

In some embodiments, the activation level of an activatable element is determined. One embodiment makes this determination by contacting a cell from a cell population with a binding element that is specific for an activation state of the activatable element. The term “binding element” includes any molecule, e.g., peptide, nucleic acid, small organic molecule which is capable of detecting an activation state of an activatable element over another activation state of the activatable element. Binding elements and labels for binding elements are shown in U.S. Pat. Nos. 8,227,202 and 8,309,306 and U.S. Ser. Nos. 12/432,720, 12/229,476, and 12/910,769.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Methods described herein may be used to detect any particular activatable element in a sample that is antigenically detectable and antigenically distinguishable from other activatable element which is present in the sample. For example, activation state-specific antibodies can be used in the present methods to identify distinct signaling cascades of a subset or subpopulation of complex cell populations and the ordering of protein activation (e.g., kinase activation) in potential signaling hierarchies. Hence, in some embodiments the expression and phosphorylation of one or more polypeptides are detected and quantified using methods described herein. In some embodiments, the expression and phosphorylation of one or more polypeptides that are cellular components of a cellular pathway are detected and quantified using methods described herein. As used herein, the term “activation state-specific antibody” or “activation state antibody” or grammatical equivalents thereof, can refer to an antibody that specifically binds to a corresponding and specific antigen. The corresponding and specific antigen can be a specific form of an activatable element. The binding of the activation state-specific antibody can be indicative of a specific activation state of a specific activatable element.

In some embodiments, the binding element is an antibody. In some embodiments, the binding element is a single or multiple antibodies. In some embodiment, the binding element is an activation state-specific antibody such as an antibody the recognizes different activation states of a marker, for example activation states can be, but are not limited to: an acetylation site, a ubiquitination site, a phosphorylation site, a methylation site, a hydroxylation site, a SUMOylation site, or a cleavage site. In some embodiment, the binding element is an activation state-specific antibody such as an antibody that recognizes non-activated states of a marker.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports to make arrays, and posses other variations such as having two different recognition sites. See U.S. Pat. No. 8,227,202 and U.S. Ser. Nos 12/432,720, 12/229,476, and 12/910,769 for more information about antibodies as binding elements.

Activation state specific antibodies can be used to detect kinase activity; however additional means for determining kinase activation are provided herein. For example, substrates that are specifically recognized by protein kinases and phosphorylated thereby are known. Antibodies that specifically bind to such phosphorylated substrates but do not bind to such non-phosphorylated substrates (phospho-substrate antibodies) can be used to determine the presence of activated kinase in a sample.

The antigenicity of an activated isoform of an activatable element can be distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of a moiety to an element, such as a phosphate moiety, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see the Cell Signaling Technology or Becton Dickinson websites) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1,Cot, NIK, Bub, Myt 1, Weel, Casein kinases, PDK1, SGK1, SGK2, SGK3, Akt1, Akt2, Akt3, p9ORsks, p70S6Kinase,Prks, PKCs, PKAs, ROCK 1, ROCK 2, Auroras, CaMKs, MNKs, AMPKs, MELK, MARKs, Chkl, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/ DNA binding proteins, Ets family transcription factors, Ets-1, Ets-2, Tel, Te12, Elk, SMADs, Rel-A (p65-NFKB), CREB, NFAT, ATF-2, AFT, Myc, Fos, Spl, Egr-1, T-bet, f3-catenin, HIFs, FOXOs, E2Fs, SRFs, TCFs, Egr-1, β-FOXO, STAT1, STAT3, STA 4, STATS, STATE, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope can be used to produce such an antibody fragment by recombinant means well known in the art.

In alternative embodiments, aromatic amino acids of protein binding elements can be replaced with other molecules. See U.S. Pat. No. 8,227,202 and U.S. Ser. Nos. 12/432,720, 12/229,476, and 12/910,769.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores or isotopes or other labels can be attached to such antibodies for use in the methods described herein.

A variety of recognitions structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In some embodiments, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. There term “chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used.

In some embodiments the binding element is a carbohydrate. As used herein the term carbohydrate can include any compound with the general formula (CH₂0)n. Examples of carbohydrates are mono-, di-, tri- and oligosaccharides, as well polysaccharides such as glycogen, cellulose, and starches.

In some embodiments the binding element is a lipid. As used herein the term lipid herein can include any water insoluble organic molecule that is soluble in nonpolar organic solvents. Examples of lipids are steroids, such as cholesterol, and phospholipids such as sphingomeylin, and fatty acyls, glycerolipids, glycerophospholipids, sphingolipids, saccharolipids, and polyketides, including tri-, di- and monoglycerides and phospholipids. The lipid can be a hydrophobic molecule or amphiphilic molecule.

Examples of activatable elements, activation states and methods of determining the activation level of activatable elements are described in U.S. Pub. No. 2006/0073474 entitled “Methods and compositions for detecting the activation state of multiple proteins in single cells” and U.S. Pub. No. 200/50112700 entitled “Methods and compositions for risk stratification” the content of which are incorporate here by reference.

Modulators

In some embodiments, the methods and composition utilize a modulator. A modulator can be an activator, a therapeutic compound, an inhibitor or a compound capable of impacting a cellular pathway. Modulators can also take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, a sample of cells is exposed to at least 2, 3, 4, 5, 6, 7, 8, 9, or 10 or more modulators. See U.S. Ser. Nos. 12/432,239 and 12/910,769 which are incorporated by reference in their entireties. See also U.S. Patent Nos. 7,695,926 and 7,381,535 and U.S. Pub. No. 2009/0269773.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%, about 0.001% to 30%, about 0.01% to 30%, about 0.1% to 30% or 1% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, mitogens, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element. A modulator can include, e.g., a psychological stressor.

In some embodiments the modulator is selected from the group consisting of growth factors, mitogens, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g., beads, plates, viral envelopes, antigen presentation molecules such as major histocompatibility complex). In some embodiments, the modulator is a physical stimuli such as heat, cold, UV radiation, and radiation. Examples of modulators include but are not limited to Growth factors, such as Adrenomedullin (AM), Angiopoietin (Ang), Autocrine motility factor, Bone morphogenetic proteins (BMPs),Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Stromal Derived Growth Factor, (SDGF), Thrombopoietin (TPO), Transforming growth factor alpha (TGF-α), Transforming growth factor beta (TGF-β), Tumour necrosis factor-alpha (TNF-α),Vascular endothelial growth factor (VEGF), Keratin Derived Growth Factor (KGF), Wnt Signaling Pathway, placental growth factor (PlGF), Fetal Bovine Somatotropin (FBS), IL-1-Cofactor for IL-3 and IL-6. Activates T cells, IL-2-T-cell growth factor. Stimulates IL-1 synthesis. Activates B-cells and NK cells, IL-3-Stimulates production of all non-lymphoid cells, IL-4-Growth factor for activated B cells, resting T cells, and mast cells,IL-5-Induces differentiation of activated B cells and eosinophils, IL-6-Stimulates Ig synthesis. Growth factor for plasma cells, and IL-7-Growth factor for pre-B cells. Cell motility factors, such as peptide growth factors, (e.g., EGF, PDGF, TGF-beta), substrate-adhesion molecules (e.g., fibronectin, laminin), cell adhesion molecules (CAMs), and metalloproteinases, hepatocyte growth factor (HGF) or scatter factor (SF), autocrine motility factor (AMF), and migration-stimulating factor (MSF). Other modulators include SDF-1α, IFN-α, IFN-γ, IL-10, IL-6, IL-27, G-CSF, FLT-3L, IGF-1, M-CSF, SCF, PMA, Thapsigargin, H₂O₂, Etoposide, Mylotarg, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L. Below are descriptions of some examples of modulators.

In one embodiment, the modulator is etoposide phosphate. Etoposide phosphate (brand names: Eposin, Etopophos, Vepesid, VP-16) can inhibit enzyme topoisomerase II. Etoposide phosphate is a semisynthetic derivative of podophyllotoxin, a substance extracted from the mandrake root Podophyllum peltatum. Etoposide can possess antineoplastic properties. Etoposide can bind to and inhibit topoisomerase II and its function in ligating cleaved DNA molecules, resulting in the accumulation of single- or double-strand DNA breaks, the inhibition of DNA replication and transcription, and apoptotic cell death. Etoposide can act primarily in the G2 and S phases of the cell cycle. See the NCI Drug Dictionary at the website:

<<http://www.cancer.gov/Templates/drugdictionary.aspx?CdrID=39207>>.

In one embodiment, the modulator is Mylotarg. Mylotarg® (gemtuzumab ozogamicin for Injection) is a chemotherapy agent composed of a recombinant humanized IgG4, kappa antibody conjugated with a cytotoxic antitumor antibiotic, calicheamicin, isolated from fermentation of a bacterium, Micromonospora echinospora subsp. calichensis. The antibody portion of Mylotarg can bind specifically to the CD33 antigen, a sialic acid-dependent adhesion protein found on the surface of leukemic blasts and immature normal cells of myelomonocytic lineage, but not on normal hematopoietic stem cells. See U.S. Pat. Nos. 7,727,968, 5,773,001, and 5,714,586.

In one embodiment, the modulator is staurosporine. Staurosporine (antibiotic AM-2282 or STS) is a natural product originally isolated in 1977 from bacterium Streptomyces staurosporeus. Staurosporine can have biological activities ranging from anti-fungal to anti-hypertensive. See e.g.,Rüegg U T, Burgess G M. (1989) Staurosporine, K-252 and UCN-01: potent but nonspecific inhibitors of protein kinases. Trends in Pharmacological Science 10 (6): 218-220. Staruosporine can be an anticancer treatment. Staurosporine can inhibit protein kinases through the prevention of ATP binding to the kinase. This inhibition can be achieved because of the higher affinity of staurosporine for the ATP-binding site on the kinase. Staurosporine is a prototypical ATP-competitive kinase inhibitor in that it can bind to many kinases with high affinity, though with little selectivity. Staurosporine can be used to induce apoptosis. One way in which staurosporine can induce apoptosis is by activating caspase-3.

In another embodiment, the modulator is AraC. Ara-C (cytosine arabinoside or cytarabine) is an antimetabolic agent with the chemical name of 1β-arabinofuranosylcytosine. Its mode of action can be due to its rapid conversion into cytosine arabinoside triphosphate, which damages DNA when the cell cycle holds in the S phase (synthesis of DNA). Rapidly dividing cells, which require DNA replication for mitosis, are therefore affected by treatment with cytosine arabinoside. Cytosine arabinoside can also inhibit both DNA and RNA polymerases and nucleotide reductase enzymes needed for DNA synthesis. Cytarabine can be used in the treatment of acute myeloid leukaemia, acute lymphocytic leukaemia (ALL) and in lymphomas where it is the backbone of induction chemotherapy.

In another embodiment, the modulator is daunorubicin. Daunorubicin or daunomycin (daunomycin cerubidine) is a chemotherapeutic of the anthracycline family that can be given as a treatment for some types of cancer. It can be used to treat specific types of leukemia (acute myeloid leukemia and acute lymphocytic leukemia). It was initially isolated from Streptomyces peucetius. Daunorubicin can also used to treat neuroblastoma. Daunorubicin has been used with other chemotherapy agents to treat the blastic phase of chronic myelogenous leukemia. On binding to DNA, daunomycin can intercalate, with its daunosamine residue directed toward the minor groove. It has the highest preference for two adjacent G/C base pairs flanked on the 5′ side by an A/T base pair. Daunomycin effectively binds to every 3 base pairs and induces a local unwinding angle of 11°, but negligible distortion of helical conformation.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the inhibitor is an inhibitor of a cellular factor or a plurality of factors that participates in a cellular pathway (e.g., signaling cascade) in the cell. In some embodiments, the inhibitor is a phosphatase inhibitor. Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

In some embodiments, a phenotypic profile of a population of cells is determined by measuring the activation level of an activatable element when the population of cells is exposed to a plurality of modulators in separate cultures. In some embodiments, the modulators include H₂O₂, PMA, SDF1α, CD4L, IGF-1, IL-7, IL-6, IL-10, IL-27, IL-4, IL-2, IL-3, thapsigardin and/or a combination thereof. For instance a population of cells can be exposed to one or more, all or a combination of the following combination of modulators: H₂O₂, PMA, SDF1α, CD40L, IGF-1, IL-7, IL-6, IL-10, IL-27, IL-4, IL-2, IL-3, or thapsigardin. In some embodiments, the phenotypic profile of the population of cells is used to classify the population as described herein.

In some embodiments, the activation level of an activatable element in a cell is determined by contacting the cell with at least 2, 3, 4, 5, 6, 7, 8, 9, or 10 modulators. In some embodiments, the activation level of an activatable element in a cell is determined by contacting the cell with at least 2, 3, 4, 5, 6, 7, 8, 9, or 10 modulators where at least one of the modulators is an inhibitor. In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, the cell signaling profile a population of cells is determined by measuring the activation level of an activatable element when the population of cells is exposed to one or more modulators. The population of cells can be divided into a plurality of samples, and the physiological status of the population can be determined by measuring the activation level of at least one activatable element in the samples after the samples have been exposed to one or more modulators. In some embodiments, the signaling profile of different populations of cells is determined by measuring the activation level of an activatable element in each population of cells when each of the populations of cells is exposed to a modulator.

Computational Identification

In some embodiments, the activation state data of a cell population is determined by contacting the cell population with one or more modulators, generating activation state data for the cell population and using computational techniques to identify one or more discrete cell populations based on the data. These techniques are implemented using computers comprising memory and hardware. In one embodiment, algorithms for generating metrics based on raw activation state data are stored in the memory of a computer and executed by a processor of a computer. These algorithms are used in conjunction with gating and binning algorithms, which are also stored and executed by a computer, to identify the discrete cell populations.

In one embodiment the invention provides for custom algorithms for compensating for an incomplete data set, or a data set that has a lack of one or more data points in the metrics based on raw activation state data. These custom algorithms compensate for the lacking data point such that a determination of diagnosis or outcome can still be determined by the compensation algorithms, which are also stored and executed by a processor of a computer.

The data can be analyzed using various metrics. For example, the median fluorescence intensity (MFI) is computed for each activatable element from the intensity levels for the cells in the cell population gate. The MFI values are then used to compute a variety of metrics by comparing them to the various baseline or background values, e.g., the unstimulated condition, autofluorescence, and isotype control. The following metrics are examples of metrics that can be used in the methods described herein: 1) a metric that measures the difference in the log of the median fluorescence value between an unstimulated fluorochrome-antibody stained sample and a sample that has not been treated with a stimulant or stained (log (MFIUnstimulated Stained)—log (MFIGated Unstained)), 2) a metric that measures the difference in the log of the median fluorescence value between a stimulated fluorochrome-antibody stained sample and a sample that has not been treated with a stimulant or stained (log (MFIStimulated Stained)—log(MFIGated Unstained)), 3) a metric that measures the change between the stimulated fluorochrome-antibody stained sample and the unstimulated fluorochrome-antibody stained sample log (MFIStimulated Stained)—log (MFIUnstimulated Stained), also called “fold change in median fluorescence intensity”, 4) a metric that measures the percentage of cells in a Quadrant Gate of a contour plot which measures multiple populations in one or more dimension 5) a metric that measures MFI of phosphor positive population to obtain percentage positivity above the background and 6) use of multimodality and spread metrics for large sample population and for subpopulation analysis.

In a specific embodiment, the equivalent number of reference fluorophores value (ERF) is generated. The ERF is a transformed value of the median fluorescent intensity values. The ERF value is computed using a calibration line determined by fitting observations of a standardized set of 8-peak rainbow beads for all fluorescent channels to standardized values assigned by the manufacturer. The ERF values for different samples can be combined in any way to generate different activation state metric. Different metrics can include: 1) a fold value based on ERF values for samples that have been treated with a modulator (ERFm) and samples that have not been treated with a modulator (ERFu), log2 (ERFm/ERFu); 2) a total phospho value based on ERF values for samples that have been treated with a modulator (ERFm) and samples from autofluorecsent wells (ERFa), log2 (ERFm/ERFa); 3) a basal value based on ERF values for samples that have not been treated with a modulator (ERFu) and samples from autofluorescent wells (ERFa), log2 (ERFu/ERFa); 4) A Mann-Whitney statistic Uu comparing the ERFm and ERFu values that has been scaled down to a unit interval (0,1) allowing inter-sample comparisons; 5) A Mann-Whitney statistic Uu comparing the ERFm and ERFu values that has been scaled down to a unit interval (0,1) allowing inter-sample comparisons; 5) a Mann-Whitney statistic Ua comparing the ERFa and ERFm values that has been scaled down to a unit interval (0,1); and 6) A Mann-Whitney statistic U75. U75 is a linear rank statistic designed to identify a shift in the upper quartile of the distribution of ERFm and ERFu values. ERF values at or below the 75th percentile of the ERFm and ERFu values are assigned a score of 0. The remaining ERFm and ERFu values are assigned values between 0 and 1 as in the Uu statistic. For activatable elements that are surface markers on cells, the following metrics may be further generated: 1) a relative protein expression metric log2(ERFstain)—log2(ERFcontrol) based on the ERF value for a stained sample (ERFstain) and the ERF value for a control sample (ERFcontrol); and 2) A Mann-Whitney statistic Ui comparing the ERFm and ERFi values that has been scaled down to a unit interval (0,1), where the ERFi values are derived from an isotype control.

The activation state data for the different markers is “gated” in order to identify discrete subpopulations of cells within the data. In gating, activation state data is used to identify discrete sub-populations of cells with distinct activation levels of an activatable element. These discrete sub-populations of cells can correspond to cell types, cell sub-types, cells in a disease or other physiological state and/or a population of cells having any characteristic in common.

In some embodiments, the activation state data is displayed as a two-dimensional scatter-plot and the discrete subpopulations are “gated” or demarcated within the scatter-plot. According to the embodiment, the discrete subpopulations may be gated automatically, manually or using some combination of automatic and manual gating methods. In some embodiments, a user can create or manually adjust the demarcations or “gates” to generate new discrete sub-populations of cells. Suitable methods of gating discrete sub-populations of cells are described in U.S. patent application Ser. No. 12/501,295, the entirety of which is incorporated by reference herein, for all purposes.

In some embodiments, the homogenous cell populations are gated according to markers that are known to segregate different cell types or cell sub-types. In a specific embodiment, a user can identify discrete cell populations based on surface markers. For example, the user could look at: “stem cell populations” by CD34+CD38- or CD34+CD33-expressing cells; memory CD4 T-lymphocytes; e.g., CD4+CD45RA+CD29^{low} cells; or multiple leukemic sub-clones based on CD33, CD45, HLA-DR, CD11b and analyzing signaling in each discrete population/subpopulation. In another alternative embodiment, a user may identify discrete cell populations/subpopulations based on intracellular markers, such as transcription factors or other intracellular proteins; based on a functional assay (e.g., dye efflux assay to determine drug transporter +cells or fluorescent glucose uptake) or based on other fluorescent markers. In some embodiments, gates are used to identify the presence of specific discrete populations and/or subpopulations in existing independent data. The existing independent data can be data stored in a computer from a previous patient, or data from independent studies using different patients.

Gating Methods

Manual or automatic gating can be used in various aspects of the present invention, such as to focus on healthy cells, cells of a certain lineage, type, or to analyze cell signaling. For example, in one embodiment gating is used to identify the healthy cell subpopulation. In one embodiment, cells are identified using Forward and Side Scatter, live cells are identified using Amine Aqua, leukemic blasts are identified using Side Scatter and CD45, and non-apoptotic leukemic blasts (Healthy P1) are identified by assaying for the absence of cleaved PARP. This embodiment focuses the analysis on healthy cells.

In another embodiment, a user will gate cells for the cell signaling component. For example, a user may analyze the signaling in subpopulations based on surface markers. For example, the user can look at: cells that have CD45, EpCam, or cytokeratin (cells that are CD45⁻/cytokeratin+/EpCam+ are epithelial cells), “stem cell populations” by CD34+CD38- or CD34+CD33-expressing cells; drug transporter positive cells; i.e. C-KIT+(SCF Receptor, CD117) cells+; FLT3+cells; CD44+cells, CD47+cells, CD123+cells, or multiple leukemic subpopulations based on CD33, CD45, HLA-DR, CD1lb and analyzing signaling in each subpopulation. In another alternative embodiment, a user may analyze the data based on intracellular markers, such as transcription factors or other intracellular proteins; based on a functional assay (e.g., dye negative “side population” aka drug transporter +cells, or fluorescent glucose uptake, or based on other fluorescent markers). In some embodiments, a gate is established after learning from a responsive subpopulation. That is, a gate is developed from one data set after finding a population that correlates with a clinical outcome. This gate can then be applied retrospectively or prospectively to other data sets. See U.S. Ser. No. 12/501,295 for an example of gating.

Both gating embodiments can be run at the same time when a user is analyzing each well/aliquot for the activatable element that relates to cell health, for example, if each well has the reagent used for detecting the activatable element related to cell health.

In some embodiments where flow cytometry is used, prior to analyzing data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification.

Each of these techniques capitalizes on the ability of flow cytometry to deliver large amounts of multiparameter data at the single cell level. For cells associated with a condition (e.g., neoplastic or hematopoetic condition), a third “meta-level” of data exists because cells associated with a condition (e.g., cancer cells) are generally treated as a single entity and classified according to historical techniques. These techniques have included organ or tissue of origin, degree of differentiation, proliferation index, metastatic spread, and genetic or metabolic data regarding the patient.

In some embodiments, methods described herein use variance mapping techniques for mapping condition signaling space. These methods represent a significant advance in the study of condition biology because they enable comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g., two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the X² test. Similarly, if two variable factors within the experiment are thought to be related, the Pearson, and/or Spearman is used to measure the degree of this relationship.

Examples of analysis for activatable elements are described in U.S. Pub. No. 2006/0073474 entitled “Methods and compositions for detecting the activation state of multiple proteins in single cells” and U.S. Pub. No. 2005/0112700 entitled “Methods and compositions for risk stratification” the content of which are incorporated herein by reference.

Labels

The methods and compositions provided herein provide binding elements comprising a label or tag in the SCNP or quality processes. A label can be a molecule that can be directly (i.e., a primary label) or indirectly (i.e., a secondary label) detected; for example a label can be visualized and/or measured or otherwise identified so that its presence or absence can be known. Binding elements and labels for binding elements are shown, e.g., in U.S. Pat. Nos. 8,227,202 and 8,309,306 and U.S. Ser. Nos. 12/432,720, 12/229,476, and 12/910,769.

A compound can be directly or indirectly conjugated to a label which provides a detectable signal, e.g., radioisotopes, fluorescers, enzymes, antibodies, particles such as magnetic particles, chemiluminescers, molecules that can be detected by mass spectrometry, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments, a label can be conjugated to a binding element.

In some embodiments, one or more binding elements are uniquely labeled. Using the example of two activation state specific antibodies, “uniquely labeled” can mean that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels can fall into four classes: a) isotopic labels, which can be radioactive or heavy isotopes; b) magnetic, electrical, thermal labels; c) colored, optical labels including luminescent, phosphorous and fluorescent dyes or moieties; and d) binding partners. Labels can also include enzymes (e.g., horseradish peroxidase, etc.) and magnetic particles. In some embodiments, the detection label is a primary label. A primary label is one that can be directly detected, such as a fluorophore. However, it is appreciated that as the technology grows any equivalent label technologies can be used with the invention.

Labels include optical labels such as fluorescent dyes or moieties. Fluorophores can be “small molecule” fluors or proteinaceous fluors (e.g., green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through the Life Technologies website.

Quantum dot labeled antibodies can be used alone or they can be employed in conjunction with organic fluorochrome-conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, activatable elements are labeled with tags suitable for Inductively Coupled Plasma Mass Spectrometer (ICP-MS) as disclosed in Tanner et al. Spectrochimica Acta Part B: Atomic Spectroscopy, 2007 March;62(3):188-195.

Detection systems based on FRET, discussed in detail below, can be used. FRET can be used in the methods described herein, for example, in detecting activation states that involve clustering or multimerization wherein the proximity of two FRET labels is altered due to activation. In some embodiments, at least two fluorescent labels are used which are members of a fluorescence resonance energy transfer (FRET) pair. In some embodiments, FRET analyses uses an automated microscope or high content cell reader to determine the level of activation states.

The methods and compositions described herein can also make use of label enzymes. A label enzyme can be an enzyme that can be reacted in the presence of a label enzyme substrate that produces a detectable product. Suitable label enzymes include but are not limited to horseradish peroxidase, alkaline phosphatase and glucose oxidase. Methods for the use of such substrates are well known in the art. The presence of a label enzyme can generally be revealed through the enzyme's catalysis of a reaction with a label enzyme substrate, producing an identifiable product. Such products may be opaque, such as the product resulting from the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes include, but are not limited to, 14C, 3H, 32P, 33P, 35S, 125I and 131I. The use of radioisotopes as labels is well known in the art.

Labels can be indirectly detected, that is, the tag is a partner of a binding pair. “Partner of a binding pair” can mean one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) can bind to a first antibody (second moiety) that can, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair can comprise a label, as described above. It will further be appreciated that a label allows for a tag to be indirectly labeled upon the binding of a binding partner comprising a label. Attaching a label to a tag that is a partner of a binding pair, as just described, can be referred to herein as “indirect labeling”.

“Surface substrate binding molecule” or “attachment tag” and grammatical equivalents thereof can mean a molecule have binding affinity for a specific surface substrate, which substrate is generally a member of a binding pair applied, incorporated or otherwise attached to a surface. Suitable surface substrate binding molecules and their surface substrates include, but are not limited to, poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate (Field et al., Mol. Cell. Biol., 8:2159-2165 (1988)); the c-myc tag and the 8F9, 3C7, 6E10, G4, B7 and 9E10 antibody substrates thereto (Evan et al., Molecular and Cellular Biology, 5:3610-3616 (1985)); and the Herpes Simplex virus glycoprotein D (gD) tag and its antibody substrate (Paborsky et al., Protein Engineering, 3(6):547-553 (1990)). In general, surface binding substrate molecules include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Detection

In practicing the methods described herein, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods described herein can be performed according to standard techniques and protocols well-established in the art.

One or more activatable elements can be detected and/or quantified by any method that detects and/or quantitates the presence of the activatable element of interest. Such methods may include flow cytometry, mass spectrometry, radioimmunoassay (RIA) or enzyme linked immunoabsorbance assay (ELISA), immunohistochemistry (IHC), immunofluorescent histochemistry with or without confocal microscopy, reversed phase assays, homogeneous enzyme immunoassays, and related non-enzymatic techniques, Western blots, Far Western, Northern Blot, Southern blot, whole cell labeling, immunoelectronmicroscopy, nucleic acid amplification, PCR, gene array, protein array, mass spectrometry, nucleic acid sequencing, next generation sequencing, patch clamp, 2-dimensional gel electrophoresis, differential display gel electrophoresis, microsphere-based multiplex protein assays, label-free cellular assays, etc. These techniques are particularly useful for modified protein parameters. Cell readouts for proteins and other cell determinants can be obtained using fluorescent or otherwise tagged reporter molecules. Flow cytometry and mass spectrometry methods are useful for measuring intracellular parameters. See e.g., U.S. Pat. No. 7,393,656 and Shults et al., Current Protocols in Immunology, 2007, 78:8.17.1-20 which are incorporated by reference in their entireties.

In some embodiments, provided herein are methods for determining an activatable element's activation profile for a single cell. The methods may comprise analyzing cells by flow cytometry on the basis of the activation level of at least two activatable elements. Binding elements (e.g., activation state-specific antibodies) can be used to analyze cells on the basis of activatable element activation level, and can be detected as described herein. Alternatively, non-binding element systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorochrome to form a detection element. Detection elements do not need to have both of the above agents, but can be one unit that possesses both qualities. These and other methods, instruments and devices are well described in U.S. Pat. Nos. 7,381,535, 7,393,656, and 8,227,202 and U.S. Ser. Nos. 10/193,462; 11/655,785; 11/655,789; 11/655,821; 11/338,957, 12/432,720, 12/229,476, and 12/910,769 (as well as the applications listed above) which are all incorporated by reference in their entireties.

In one embodiment, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions described herein, it will recognized that different types of fluorescent monitoring systems, e.g., cytometric measurement device systems, can be used. In some embodiments, flow cytometric systems are used or systems dedicated to high throughput screening, e.g., 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361. Commercial instruments are available through Becton Dickinson and Beckman Coulter, among others.

Fluorescence in a sample can be measured using a fluorimeter. In general, excitation radiation, from an excitation source having a first wavelength, passes through excitation optics. The excitation optics deliver the apporiate radiation wavelength to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for visual presentation on a screen or in the form of a paper or electronic report. In general, known robotic systems and components can be used in conjunction with the invention.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g., size, granularity, or fluorescent intensity.

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, can be measured. The presence and intensity of the signals corresponding to the labels on the binding element indicates the level of activation of the activatable element on that cell (Tanner et al. Spectrochimica Acta Part B: Atomic Spectroscopy, 2007 March;62(3):188-195.). See also, U.S. Pub. No. 2012/0056086, 2011/0253888, 2009/0134326, and 2011/0024615 which are incorporated by reference in their entireties.

The detecting, sorting, or isolating step of the methods described herein can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods described herein (see e.g., W099/54494 and U.S. Pub. No. 2001/0006787, each expressly incorporated herein by reference).

In some embodiments, a FACS cell sorter (e.g., a FACSVantage™ Cell Sorter, Becton Dickinson Immunocytometry Systems, San Jose, Calif.) is used to sort and collect cells based on their activation profile (positive cells) in the presence or absence of an increase in activation level of an activatable element in response to a modulator. Other flow cytometers that are commercially available include the LSR II and the Canto II both available from Becton Dickinson. Other flow cytometers include the Attune Acoustic Cytometer (Life Technologies, Carlsbad Calif.) and the CyTOF (DVS Sciences, Sunnyvale, Calif.). See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g., antibodies) directed against a specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ manual, with particular reference to sections 3-11 to 3-28 and 10-1 to 10-17, which is hereby incorporated by reference in its entirety. See the patents, applications and articles referred to, and incorporated above for detection systems.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex manual which is hereby incorporated in its entirety.

In some embodiments, methods for the determination of a receptor element activation state profile for a single cell are provided. The methods comprise providing a population of cells and analyzing the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiments, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the methods described herein also provide for the ordering of element clustering events in signal transduction. Particularly, the methods described herein allow the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

Provided herein is a method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the methods described herein allow the individual evaluation of cells to allow true differences to be identified in a significant way.

Thus, provided herein are methods of distinguishing cellular subsets within a larger cellular population. As outlined herein, these cellular subsets often exhibit altered biological characteristics (e.g., activation levels, altered response to modulators) as compared to other subsets within the population. For example, as outlined herein, the methods described herein allow the identification of subsets of cells from a population such as primary cell populations, e.g., peripheral blood mononuclear cells that exhibit altered responses (e.g., response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such a peripheral blood mononuclear cells, or naive and memory lymphocytes.

When necessary cells are dispersed into a single cell suspension, e.g., by enzymatic digestion with a suitable protease, e.g., collagenase, dispase, etc; and the like. An appropriate solution is used for dispersion or suspension. Such solution will generally be a balanced salt solution, e.g., normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be fixed, e.g., with 3% paraformaldehyde, and are usually permeabilized, e.g., with ice cold methanol; HEPES-buffered PBS containing 0.1% saponin, 3% BSA; covering for 2 min in acetone at -200° C.; and the like as known in the art and according to the methods described herein.

In one embodiment, a methanol dispensing instrument is used to permeabilize the cells. It is important to ensure that the correct volume of methanol is being dispensed into the wells, otherwise the labeling reagents will not have access to their targets. To ensure that the appropriate amount of methanol is dispensed, the dispenser is charged beforehand with methanol or is charged with methanol either manually or automatically.

The methanol dispensing heads in the instrument can be stored with methanol or air in the dispensing channels. Air can be drawn through the dispensing heads, then an alcohol solution and then stored air dried or with methanol. Upon reuse of the instrument or any restart of the process, the dispensing heads are recharged with methanol. A bleeder valve can be used to fill up the head with the correct amount of methanol. In one embodiment, the instrument dispenser is charged by flushing several methanol washes through the dispenser head. In one embodiment, 2, 3, 4, 5, 6, washes are used to fill and clean the head.

In some embodiments, the present invention uses platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. One embodiment uses microtiter plates and reference will be made to this embodiment as a representative of those articles that can contain samples to be analyzed.

In some embodiments, one or more cells are contained in a well of a 96 well plate or other commercially available multiwell plate. In an alternate embodiment, the reaction mixture or cells are in a cytometric measurement device. Other multiwell plates useful in the methods described herein include, but are not limited to 384 well plates and 1536 well plates. Still other vessels for containing the reaction mixture or cells and useful for the methods described herein will be apparent to the skilled artisan. Methods to automate the analysis are shown in U.S. Ser. No. 12/606,869 which is hereby incorporated by reference in its entirety.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

As will be appreciated by one of skill in the art, the instant methods and compositions find use in a variety of other assay formats in addition to flow cytometry analysis. For example, DNA microarrays are commercially available through a variety of sources (Affymetrix, Santa Clara Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array.

In some embodiments, such a chip comprises a multiplicity of element activation state binding elements, and is used to determine an element activation state profile for elements present on the surface of a cell.

In some embodiments, the methods and compositions described herein can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g., EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described herein.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods described herein include the use of liquid handling components. The liquid handling systems can include robotic systems comprising any number of components. In addition, any or all of the steps outlined herein may be automated; thus, for example, the systems may be completely or partially automated. See U.S. Ser. Nos. 12/606,869 and 12/432,239.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods described herein include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices described herein. The general interaction between a central processing unit, a memory, input/output devices, and a bus is known in the art. Thus, a variety of different procedures, depending on the experiments to be run, are stored in the CPU memory. See FIGS. 20 and 21 of U.S. Ser. No. 12/688,851 for a computer system useful for one embodiment of the present invention.

These robotic fluid handling systems can utilize any number of different reagents, including buffers, reagents, samples, washes, assay components such as label probes, etc.

Any of the steps above can be performed by a computer program product that comprises a computer executable logic that is recorded on a computer readable medium. For example, the computer program can execute some or all of the following functions: (i) exposing reference population of cells to one or more modulators, (ii) exposing reference population of cells to one or more binding elements, (iii) detecting the activation levels of one or more activatable elements, (iv) characterizing one or more cellular pathways, (v) classifying one or more cells into one or more classes based on the activation level (vi) determining cell health status of a cell, (vii) determining the percentage of viable cells in a sample; (viii) determining the percentage of healthy cells in a sample; (ix) determining a cell signaling profile; (x) adjusting a cell signaling profile based on the percentage of healthy cells in a sample; (xi) adjusting a cell signaling profile for an individual cell based on the health of the cell; (xii) excluding or including a cell or population of cells in a cell signaling analysis based on the health of the cell or population of cells; (xiii) assaying for one or more cell health markers; and/or (xiv) assaying for one or more apoptosis and/or necrosis markers. One embodiment of the invention employs one or more of the above functions. Other embodiments employ 2, 3, 4, 5, 6, 7, 8, 9, 10 or more of the above functions.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g., microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology Feb. 2008. Methods for the analysis of multiple parameters are well known in the art. See U.S. Ser. Nos. 11/338,957, 12/910,769, 12/293,081, 12/538,643, 12/501,274 and PCT/2011/48332 for more information on analysis. See U.S. Ser. No. 12/501,295 for gating analysis.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g., tumor cells). See also U.S. Ser. No. 12/501,295 for visualization tools.

The patients are stratified based on nodes that inform the clinical question using a variety of metrics. To stratify the patients between those patients with No Response (NR) versus a Complete Response (CR), a prioritization of the nodes can be made according to statistical significance (such as p-value from a t-test or Wilcoxon test or area under the receiver operator characteristic (ROC) curve) or their biological relevance.

In some embodiments the automated methods of the present invention are enacted on and/or by using computer systems. Examples of computer systems of the invention are described below. Variations on the described computer systems are possible so long as they provide an appropriate and compatible platform for the methods of the invention. An example of computer system of the invention is illustrated in See FIGS. 20 and 21 of U.S. Ser. No. 12/688,851 for a computer system useful for one embodiment of the present invention.

The computer system 2100 illustrated in FIG. 21 of U.S. Ser. No. 12/688,851 may be understood as a logical apparatus that can read instructions from media 2111 and/or a network port 2105, which can optionally be connected to server 2109 having fixed media 2112. The system, such as shown in FIG. 21 can include a central processing unit (CPU) 2101, disk drives 2103, optional input devices such as keyboard 2115 and/or mouse 2116 and optional monitor 2107. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 2122 as illustrated in FIG. 21.

The method or system may also be practiced in distributed computing environments where tasks are performed by remote processing devices that are linked through a communications network. FIG. 20, an exemplary system for implementing the method or system includes a general purpose computing device in the form of a computer 2002. Components of computer 2002 may include, but are not limited to, a processing unit 2004, a system memory 2006, and a system bus 2008 that couples various system components including the system memory to the processing unit 2004. Non-limiting examples of processors include: Intel XeonTM processor, AMD Opteron™ processor, Samsung 32-bit RISC ARM 1176JZ(F)-S v1.0™ processor, ARM Cortex-A8 Samsung S5PC100M processor, ARM Cortex-A8 Apple A4™ processor, Marvell PXA 930™ processor, or a functionally-equivalent processor. Multiple threads of execution can be used for parallel processing. In some aspects of the invention, multiple processors or processors with multiple cores can also be used, whether in a single computer system, in a cluster, or distributed across systems over a network comprising a plurality of computers, cell phones, and/or personal data assistant devices.

Computer 2002 typically includes a variety of computer readable media. Computer readable media includes both volatile and nonvolatile media, removable and non-removable media and a may comprise computer storage media. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices.

The system memory 2006 includes computer storage media in the form of volatile and/or nonvolatile memory such as read only memory (ROM) 2010 and random access memory (RAM) 2012. A basic input/output system 2014 (BIOS), containing the basic routines that help to transfer information between elements within computer 2002, such as during start-up, is typically stored in ROM 2010. RAM 2012 typically contains data and/or program modules that are immediately accessible to and/or presently being operated on by processing unit 2004. FIG. 20 illustrates operating system 2032, application programs 2034 such as sequence analysis, probe selection, signal analysis, gating algorithms, compensation algorithms, and cross-hybridization analysis programs, other program modules 2036, and program data 2038.

The computer 2002 may also include other removable/non-removable, volatile/nonvolatile computer storage media. By way of example only, FIG. 20 illustrates a hard disk drive 2016 that reads from or writes to non-removable, nonvolatile magnetic media, a magnetic disk drive 2018 that reads from or writes to a removable, nonvolatile magnetic disk 2020, and an optical disk drive 2022 that reads from or writes to a removable, nonvolatile optical disk 2024 such as a CD ROM or other optical media. Other removable/non-re-movable, volatile/nonvolatile computer storage media that can be used in the exemplary operating environment include magnetic tape cassettes, flash memory cards, digital versatile disks, digital video tape, solid state RAM, solid state ROM, and the like. The hard disk drive 2016 is typically connected to the system bus 2008 through a non-removable memory interface such as interface 2026, and magnetic disk drive 2018 and optical disk drive 2022 are typically connected to the system bus 2008 by a removable memory interface, such as interface 2028 or 2030.

The drives and their associated computer storage media discussed above and illustrated in FIG. 20, provide storage of computer readable instructions, data structures,

Program modules and other data for the computer 2002. In FIG. 20, for example, hard disk drive 2016 is illustrated as storing operating system 2032, application programs 2034, other program modules 2036, and program data 2038. Non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention.

An operator may enter commands and information into the computer 2002 through input devices such as a keyboard 2040 and a mouse, trackball or touch pad 2042. These and other input devices are often connected to the processing unit 2004 through a operator input interface 2044 that is coupled to the system bus, but may be connected by other interface and bus structures, such as a parallel port or a universal serial bus (USB). A monitor 2058 or other type of display device is also connected to the system bus 2008 via an interface, such as a video interface or graphics display interface 2056. In addition to the monitor 2058, computers may also include other peripheral output devices such as speakers (not shown) and printer (not shown), which may be connected through an output peripheral interface (not shown).

The computer system 2002 can be integrated into an analysis system, such as a analysis system reader for example as flow cytometry system, plate reader, high-content cell analyzer, IHC reader, automated microscope or alternatively the data can be generated by an analysis system and then subsequently be imported or uploaded into the computer system using various means known in the art.

The computer system 2002 may operate in a networked environment using logical connections to one or more remote computers or analysis systems. The remote computer may be a personal computer, a server, a router, a network PC, a peer device or other common network node, and typically includes many or all of the elements described above relative to the computer 2002. The logical connections depicted in FIG. 20 include a local area network (LAN) 2048 and a wide area network (WAN) 2050, but may also include other networks. Such networking environments are commonplace in offices, enterprise-wide computer networks, intranets and the Inter-net.

When used in a LAN networking environment, the computer 2002 is connected to the LAN 2048 through a network interface or adapter 2052. When used in a WAN networking environment, the computer 2002 typically includes a modem 2054 or other means for establishing communications over the WAN 2050, such as the Internet. The modem 2054, which may be internal or external, may be connected to the system bus 2008 via the operator input interface 2044, or other appropriate mechanism. In a networked environment, program modules depicted relative to the computer 2002, or portions thereof, may be stored in the remote memory storage device.

In some embodiments, methods include use of one or more computers in a computer system. In some embodiments, the computer system is integrated into and is part of an analysis system, gene chip reader, cell plate reader, high-content cell reader, automated microscope, or a flow cytometry machine, robotic liquid handler or other robotic laboratory equipment. In other embodiments, the computer system is connected to or ported to an analysis system. In some embodiments, the computer system is connected to an analysis system by a network connection.

The computer systems and the secondary computer may thus link laboratory instruments to the systems and methods of the invention for direct input into the (method) system. For example, an automated laboratory IHC reader, flow cytometry or any other cell analyzer known in the art for generating data may interact with the systems of the invention to provide tools for local visualization and manipulation of the data generated without requiring an operator to upload the data. A direct link can be used to upload the data on demand of the operator or it can be used to upload data after a particular cycle occurs. The visualization and manipulation tools can utilize databases containing the method and intensity thresholds, gating threshold, modulator thresholds stored remotely, for example in a network.

System Interface

Established, commonly accepted device interface standards may be used to ease automation and integration of systems. In some aspects of the invention, the Standardization in Lab Automation (SiLA) device interface standard may be used to integrate labotory equipment such as a gene chip reader, slide reader, cell plate reader, robotic liquid handler, high-content cell reader, automated microscope or a flow/mass cytometry machine. By grouping devices of the same functionality device classes can be created. SiLA common command sets define commands for these device classes. SiLA defines the command names, the number of parameters and their names as well as the return data. Since commands and parameters are described in the WSDL documentation tag of the commands web service, a process management software can automatically generate a list available commands for each device. Standards may focus on defining interfaces and protocols to interconnect any lab equipment to any control application, for example a SiLA enabled control application. In some aspects of the invention, devices can be controlled through a common command set, such as the SiLA common command set. Standards may be applied to custom systems. In some cases, standards may be incorporated to commercially available components of a system that can be obtained modularly from one or more supplier's laboratory equipment.

In some embodiments, a software wrapper may translate native device drivers into a standard command structure, for example a SiLA compatible command structure. Software wrappers may be implemented without changing the hardware.

In some embodiments, interface converter hardware with specific protocol converter software is connected to the native hardware interface, to encapsulate the device, providing high compatibility.

Handling and Storage of Data

The system allows for storage of data from experiment conducted as well as the analysis of biological data. The invention provides for a system allows for storage of data from experiments into a structured database that will provide information about experiments in the form of query and or electronic or paper reports. To use the system a operator will obtain a biological data set or multiple data sets that can be stored in a database. Typically the operator will be a clinical scientist/operator who performs a biological assay which results in a data set. The biological data can be compared with stored data which is extracted or outputted from software. For example the data can be a data file that is generated from a biological assay. For example the data can be a data file that is generated from a high-content cell screening experiment. For example the data can be a data file that is generated from a plate or slide reader. For example the data can be a data file that is generated from mass spectrometry experiment, antibody-based experiment such as protein array, FRET-analysis, high-content cell reader, tissue microarray, 2D gel analysis, flow cytometry and/or ELISA. The data sets can be related to diagnostics or clinical data or the data sets can be generated for basic scientific research.

The system can use data entirely supplied by the user, but in preferred embodiments the system additionally includes data from sources other than the user. The system can then allow the operator to determine how the operator provided data is related to the data from other sources, and/or how the operator supplied data is related to itself in light of the data from other sources. In various aspects of the invention, the content of the data supplied by someone other than the operator comprises data related to protein expression, protein modification, protein-protein interaction, protein localization or drug/modulator response. The data sets can be related to diagnostics or clinical data or the data sets can be generated from basic scientific research. In some embodiments the data supplied by someone other than the operator comprises information extracted biological experiments either manually or automatically the system can use a structured database to organize the data.

II. Uses for Methods

The methods described herein are suitable for any condition for which a correlation between the cell signaling profile of a cell and the determination of a disease predisposition, diagnosis, prognosis, and/or course of treatment in samples from individuals may be ascertained. In some embodiments, the methods described herein are directed to methods for analysis, drug screening, diagnosis, prognosis, and for methods of disease treatment and prediction. In some embodiments, the methods described herein comprise methods of analyzing experimental data. In some embodiments, the cell signaling profile of a cell population comprising a genetic alteration is used, e.g., in diagnosis or prognosis of a condition, patient selection for therapy, e.g., using some of the agents identified herein, to monitor treatment, modify therapeutic regimens, and/or to further optimize the selection of therapeutic agents which may be administered as one or a combination of agents. In some embodiments, the cell population is not associated and/or is not causative of the condition. In some embodiments, the cell population is associated with the condition but it has not yet developed the condition. The cell signaling profile of a cell population can be determined by determining the activation level of at least one activatable element in response to at least one modulator in one or more cells belonging to the cell population. The cell signaling profile of a cell population can be determined by adjusting the profile based on the presence of unhealthy cells in a sample.

In one embodiment, the methods described herein can be used to prevent disease, e.g., cancer by identifying a predisposition to the disease for which a medical intervention is available. In another embodiment, an individual afflicted with a condition can be identified and treated. In another embodiment, methods are provided for assigning an individual to a risk group. In another embodiment, methods of predicting the increased risk of relapse of a condition are provided. In another embodiment, methods of predicting the risk of developing secondary complications are provided. In another embodiment, methods of choosing a therapy for an individual are provided. In another embodiment, methods of predicting the duration of response to a therapy are provided. In another embodiment, methods are provided for predicting a response to a therapy. In another embodiment, methods are provided for determining the efficacy of a therapy in an individual. In another embodiment, methods are provided for determining the prognosis for an individual.

The cell signaling profile of a cell population can serve as a prognostic indicator of the course of a condition, e.g. whether a person will develop a certain tumor or other pathologic conditions, whether the course of a neoplastic or a hematopoietic condition in an individual will be aggressive or indolent. The prognostic indicator can aid a healthcare provider, e.g., a clinician, in managing healthcare for the person and in evaluating one or more modalities of treatment that can be used. In another embodiment, the methods provided herein provide information to a healthcare provider, e.g., a physician, to aid in the clinical management of a person so that the information may be translated into action, including treatment, prognosis or prediction.

In some embodiments, the methods described herein are used to screen candidate compounds useful in the treatment of a condition or to identify new druggable targets.

In another embodiment, the cell signaling profile of a cell population can be used to confirm or refute a diagnosis of a pre-pathological or pathological condition.

In instances where an individual has a known pre-pathologic or pathologic condition, the cell signaling profile of the cell population can be used to predict the response of the individual to available treatment options. In one embodiment, an individual treated with the intent to reduce in number or ablate cells that are causative or associated with a pre-pathological or pathological condition can be monitored to assess the decrease in such cells and the state of a cellular network over time. A reduction in causative or associated cells may or may not be associated with the disappearance or lessening of disease symptoms. If the anticipated decrease in cell number and/or improvement in the state of a cellular network do not occur, further treatment with the same or a different treatment regimen may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regime can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level, thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another cell population(s) that are associated with a good prognosis. If a beneficial population of cells is observed, measures can be taken to further increase their numbers, such as the administration of growth factors. Alternatively, individuals may be monitored for the appearance or increase in cell number of another cells population(s) associated with a poor prognosis. In such a situation, renewed therapy can be considered including continuing, modifying the present therapy or initiating another type of therapy.

In one embodiment of the invention, the present method is employed on tumor or neoplastic cells. In one embodiment the cells are from solid tumors. The solid tumor may be any solid tumor amenable to sampling for direct or indirect analysis; solid tumors include but are not limited to head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma. In one embodiment, the tumor or neoplastic condition can be a blood or hematopoetic condition. Hematopoietic conditions include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the tumor or neoplastic cells are from a hematopoietic condition. Examples are: non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

Conditions and Indications

The methods described herein can be applicable to any cancerous condition in an individual involving, indicated by, and/or arising from, in whole or in part, an altered cell signaling profile in cells. In some embodiments, the cell signaling profile of a cell is determined by measuring characteristics of at least one cellular component of a cellular pathway in cells from different populations (e.g., different cell networks). Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253; Weinberg, Chapter 6, The biology of Cancer, 2007; and Blume-Jensen and Hunter, Nature, vol 411, 17 May 2001, p 355-365); See also U.S. Ser. No. 12/910,769. A condition involving or characterized by altered cell signaling profile can be readily identified, for example, by determining the state of one or more activatable elements in cells from different populations, as taught herein.

In some embodiments, the neoplastic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

In some embodiments, the neoplastic or hematopoietic condition is a B-cell or B cell lineage derived disorder. Examples of B-cell or B cell lineage derived neoplastic or hematopoietic condition include but are not limited to Chronic Lymphocytic Leukemia (CLL), B-lymphocyte lineage leukemia, B-lymphocyte lineage lymphoma, Multiple Myeloma, and plasma cell disorders, including amyloidosis and Waldenstrom's macroglobulinemia.

Other conditions can include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA April 28, 1998;95(9):5246-50), increased IGF-1 expression in prostate cancer (Schaefer et al., Science October 9 1998, 282: 199a), EGFR overexpression and resulting ERK activation in glioma cancer (Thomas C Y. Int J Cancer Mar. 10, 2003;104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

III. Kits

In some embodiments, kits are provided. Kits may comprise one or more of the state-specific binding elements described herein, such as phospho-specific antibodies. A kit may also include other reagents, such as modulators, fixatives, containers, plates, buffers, therapeutic agents, instructions, and the like. A kit can be used to assay for one or more cell health markers. A kit can be used to assay for one or more markers of apoptosis and/or necrosis.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHPT, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCy1, PLCy 2, STAT1, STAT3, STAT4, STAT5, STAT6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, pl4Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bc1-2, Mcl-1, Bcl-XL, Caspase 2, Caspase 3, Caspase 6, Caspase 7, Caspase 8, Caspase 9, IAPB, Smac, Fodrin, Actin, Src, Lyn, Fyn, Lck, NIK, IκB, p65(Re1A), IKKa, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, β-catenin, CrkL, GSK3α, GSK3β, and FOXO. In some embodiments, the kit comprises one or more of the phospho-specific antibodies specific for the proteins selected from the group consisting of Erk1, Erk2, Syk, Zap70, Lck, Btk, BLNK, Cbl, PLCγ2, Akt, Re1A, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCy, PLCy 2, STAT1, STAT3, STAT4, STAT5, STAT6, CREB, Lyn, p-S6, Cbl, NF-kB, GSK3β, CARMA/Bc110 and Tcl-1.

One embodiment uses a kit having the following reagents: Phenotyping, DNA content, and signaling reagents. Specifically, the kit includes Phenotyping, including CytoKeratin FITC, EpCAM PerCP-Cy5.5, CD45 PE-Cy7; DNA Content dye, such a DAPI; Apoptosis markers, including cPARP AF700; and Intracellular Signaling markers including, pERK PE, pAKT AF647.

The state-specific binding element can be conjugated to a solid support and to detectable groups directly or indirectly. The reagents can also include ancillary agents such as buffering agents and stabilizing agents, e.g., polysaccharides and the like. The kit can further include, e.g., other members of the signal-producing system of which system the detectable group is a member (e.g., enzyme substrates), agents for reducing background interference in a test, control reagents, apparatus for conducting a test, and the like. The kit can be packaged in any suitable manner, typically with all elements in a single container along with a sheet of printed instructions for carrying out the test.

Such kits can enable the detection of activatable elements by sensitive cellular assay methods, such as IHC (immunohistochemistry) and flow cytometry, which are suitable for the clinical detection, prognosis, and screening of cells and tissue from patients, such as leukemia patients, having a disease involving altered pathway signaling.

Such kits can comprise one or more therapeutic agents. The kit can further comprise a software package for data analysis of cell signaling profiles, which can include reference profiles for comparison with the test profile.

Such kits can also information, such as scientific literature references, package insert materials, clinical trial results, and/or summaries of these and the like, which indicate or establish the activities and/or advantages of the composition, and/or which describe dosing, administration, side effects, drug interactions, or other information useful to a health care provider. Such information can be based on the results of various studies, for example, studies using experimental animals involving in vivo models and studies based on human clinical trials. Kits described herein can be provided, marketed and/or promoted to health care providers, including physicians, nurses, pharmacists, formulary officials, and the like. Kits can also, in some embodiments, be marketed directly to the consumer.

### IV. EXAMPLES

### Example 1

### Automated Quality Control Process for Single Cell Network Profiling Used in Diagnosis, Prognosis or Drug Development

One embodiment of the methods described herein is applied to single cell network profiling which is referenced above. Generally, the process involves treating or inducing cells with a modulator, a labeling step, and a flow cytometry step. The treatment step with a modulator step can start with previously frozen cells and end with cells fixed and permeabilized with a compound, such as methanol. Then the cells can be stained with an antibody directed to a particular activated protein of interest and then analyzed using a flow cytometer. These general steps are disclosed in some references referred to above, including U.S. Ser. Nos. 61/350,864 and U.S. Pat. No. 8,227,202.

Cell Thawing, Ficoll Density Gradient Separation, and Live/Dead Labeling:

Sample Cells and standard control cells are thawed in a 37° C. water bath in cryovials. Once the cells are thawed, 1 mL of pre-warmed thaw buffer (RPMI+60% FBS) is added dropwise to the cryovials and then the entire contents of the cryovials are transferred to a 15 mL conical tube. The volume of each sample is brought up to 12 mL by adding the appropriate volume of thaw buffer. The 15 mL tubes are then capped and inverted 3 times.

A ficoll density gradient separation is then performed by underlaying 2 mL of ambient temperature ficoll using a Pasteur pipette on the samples. Next, the tubes are centrifuged at 400×g for 30 minutes at room temperature, the “buffy coat” aspirated, and the mononuclear cell layer transferred to a new 15 mL conical tube containing 9 mL thaw buffer. The cell layers are centrifuged at 400×g for 5 minutes, the liquid aspirated, the cell pellet gently resuspended. Subsequently, 10 mLs ambient temperature RPMI +1% FBS is added to the cell pellets and the cells centrifuged at 400×g for 5 minutes. The cell pellet is resuspended in 1 mL PBS and, if necessary, cell clumps removed by filtering (Celltrics filters) or by pipetting.

1 mL of PBS/Amine Aqua solution is added to the samples, the samples are mixed thoroughly by pipetting, and are incubated in a 37° C. water bath for 15 minutes.

After 15 minute incubation, 1 ml RPMI+10% FBS is added to the samples, a 150 μL aliquot removed from each sample and is placed in a 12×75 mm FACSTube. A cell count is performed on the AcT10 hematology analyzer. 5 mL RPMI+10% FBS are added to the samples, the cells are centrifuged at 400×g for 5 minutes, the liquid is aspirated, and the cells are resuspended at 1.25×10⁶ cells/mL in RPMI+10% FCS. The cells are kept in a 37° C. water bath until ready to array in deep-well plates.

Treatment of Cells with Modulators:

A concentration for each modulator (e.g., stimulant) that is five fold (5×) more than the final concentration is prepared using Media A as diluents. The 5× modulators (e.g., stimulants) are arrayed in a standard 96 well v-bottom plate that corresponds to the well on the plate with the cells to be stimulated. Fixative is prepared by dilution of stock 10% to 32% paraformaldehyde (typically 32%) with PBS to a concentration that is 2.4%, then placed in a 37° C. water bath. Once the plated cells have completed their incubation, the plate(s) are taken out of the incubator and placed in a 37° C. water bath next to the pipette apparatus. Prior to addition of stimulant, each plate of cells is taken from the water bath and gently swirled to resuspend any settled cells. The stimulant is pipetted into the cell plate, which is then held over a vortexer set to “7” and mixed for 5 seconds, and followed by the return of the deep well plate to the water bath. Modulation times can include 5, 10, and 15 minutes in a 37° C. water bath. For longer incubation times, or for assays measuring induced apoptosis, cells are modulated for 6-72 h and restained with Amine Aqua viability dye prior to the fixation steps below.

Fixing Cells and Cell Permeabilzation:

Fixation is performed using approximately 2.4% paraformaldehyde (Electron Microscopy Sciences, Hatfield, Pa.) diluted in PBS and is added to cells for a final concentration of 1.6%. The cells are pipetted up and down three times to mix and incubated for 10 minutes at 37° C. Next, the plates are centrifuged at 1000×g for 5 minutes at room temperature, the liquid aspirated from the cell pellets, and cell pellets are resuspended and the cells are permeabilized with 200 μL/well 100% ice cold methanol (SigmaAldrich), is added while vortexing. Cell plates are then covered with a foil seal and stored overnight at −80° C.

Surface and Intracellular Cell Labeling:

Plates from −80° C. storage are centrifuged at 1000×g for 5 minutes at room temperature, the supernatant is aspirated, and the cell pellet is disrupted by vortexing for 10 seconds and a speed of “3000.” Then, the cell pellets are washed two times with 1 mL FACS Buffer (PBS 0.5% BSA, 0.05% NaN₃), and are incubated at room temperature at room temperature, centrifuged at 1000×g for 5 minutes at room temperature, supernatant aspirated, and the cell disrupted by vortexing as above.

Next, 20 μL of antibody cocktail is added to each well in the cell plate, the mixture is pipetted up and down 3 times to mix, and the cells are incubated at 25° C. for 1 hr or 4° C. overnight (16 hours). After incubation, cells are washed twice by the same procedure as above.

Subsequently, 10 ul of secondary antibody mix is added the cells, the mixture is pipetted up and down three times to mix, the plate covered, and the cells incubated at 25° C. for 30 minutes. After incubation, cells are washed twice by the same procedure as above.

Label Stabilization and Preparation for Flow Cytometry:

The labeled cells are then stabilized by addition 1 mL of 1.6% PFA, the cells are covered and incubated at room temperature for 5 minutes. The cells are then centrifuged at 1000×g for 5 minutes, the supernatant is aspirated, the cell pellet is disrupted by vortexing as above, the cells are resuspended in 100μL A FACS Buffer, and are mixed by pipetting up and down 4 times. The mixed cells are transferred to a 96-well u-bottom plate and 100 μL of pre-diluted (40 μL into 1 mL of FACS Buffer) Sphero Rainbow 8-peak fluorescent beads to all wells. The plates are sealed with foil and placed at 4° C. in the dark until ready for acquisition on the flow cytometer.

Quality checks for each step in the process including the addition of beads, addition of cell lines, addition of stain controls, and the monitoring of cell surface markers. Comparing the standard cell data generated from each step of the assay to a preset range of acceptable values/thershold using a computer processor system and computer program readable medium to determine is the acceptable threshold were met and generating a report for the operator scientist or clinical lab scientist.

### Example 2

### Qualitity Control of Modulation and Signal Detection

Two cell lines, GDM-1 and RS;411, were used in an assay similar to that shown in Example 1 above. The cell lines were run alongside AML test samples. The cell lines were tested over multiple days with different batches of antibody reagents.

The cell line node-metric values from each plate are plotted in chronological order of the plates. The following process was used to inspect the cell line data and exclude data points from a plate if the pre-defined range of expression is not met: Uᵤ metrics for each node are computed to assess the level of modulation (or inhibition) of the intra-cellular markers in the cell lines. The Uᵤ value for each node for each cell line was compared to the inner and outer target values computed using data from previous studies. The upper and lower limits are defined as follow: U₁=M−n*S and Uᵤ=M+n*S Where MM is the median value for the repeats, and SS is the median absolute deviation (MAD), a robust estimate of the standard deviation for the repeats and nn is either 2 (inner limits) or 3 (outer limits). At the end of data acquisition for each batch, the cell line data was uploaded to the cell line monitoring database and visually inspected in Tableau software.

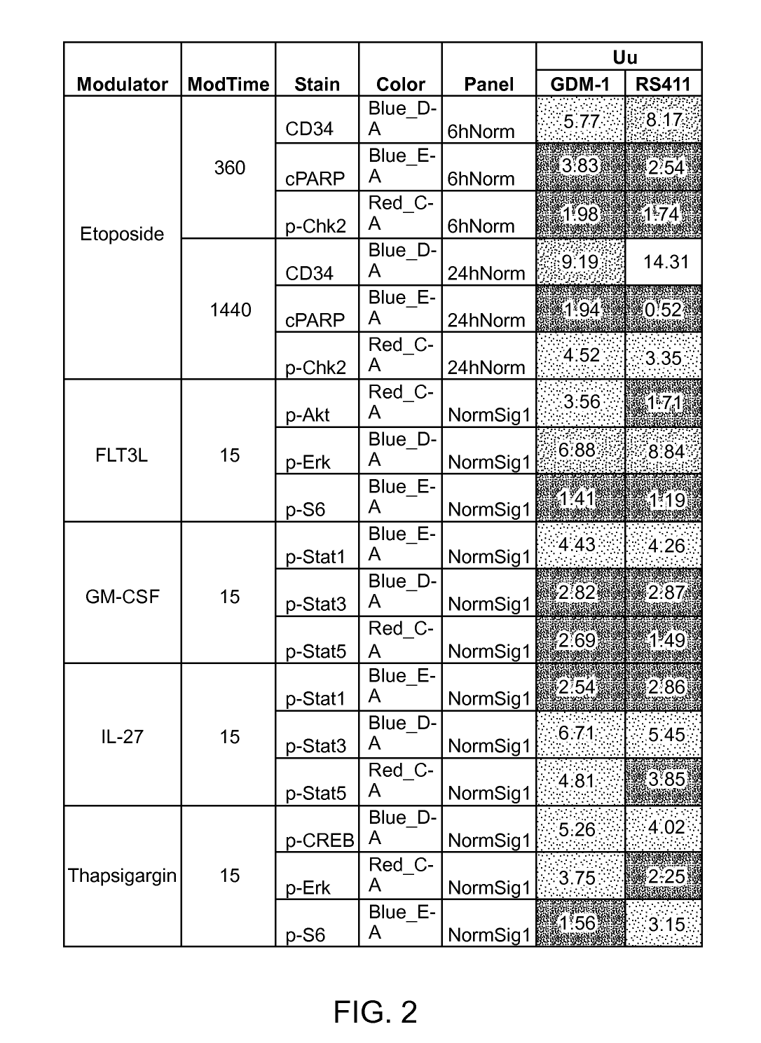

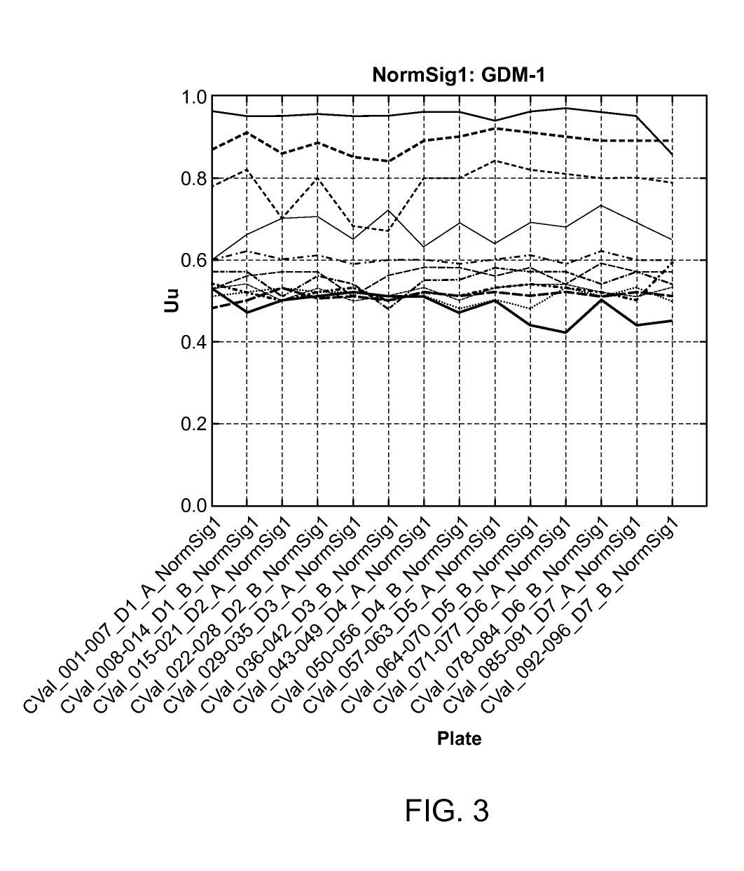

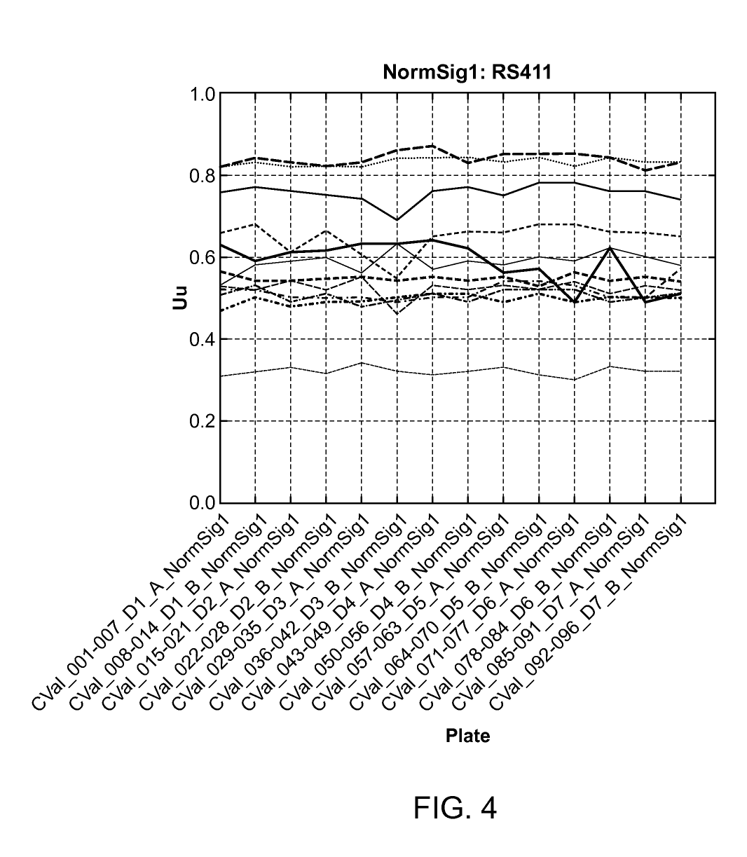

FIGS. 2-4 show the coefficient of variation for the cell lines. 35 of 36 (97%) of the cell lines tested under 10% and 28 of 36 (78%) tested under 5%. These results show that the process was working throughout the whole experiment and that much of the variation was within 10%.

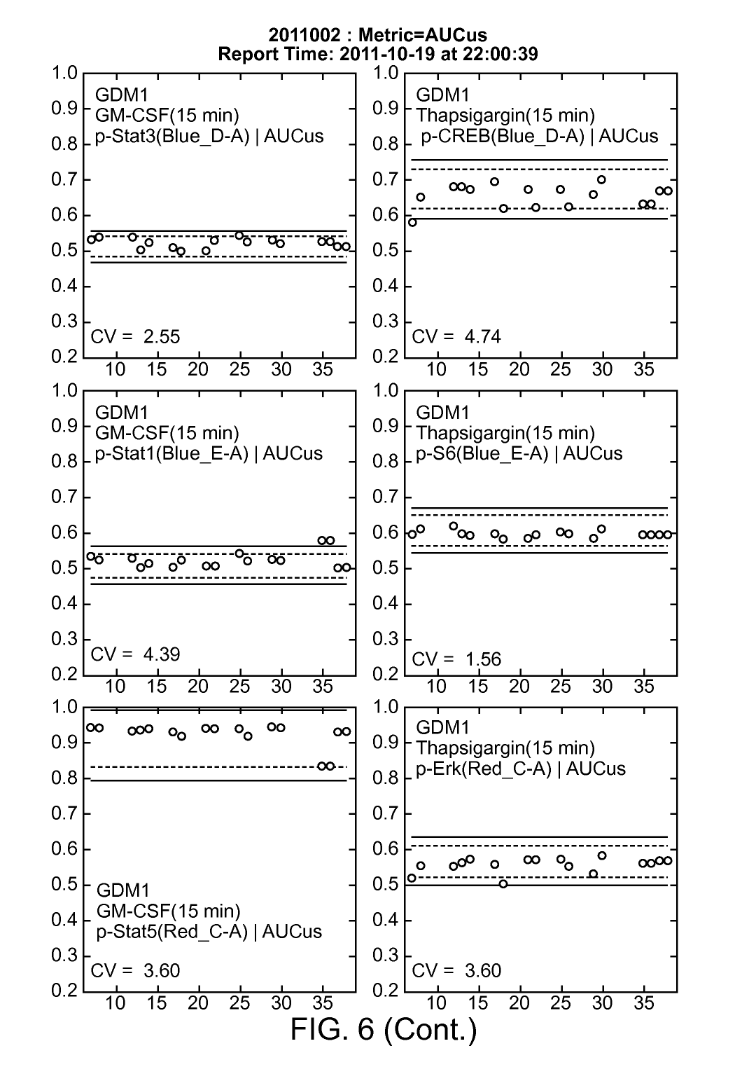

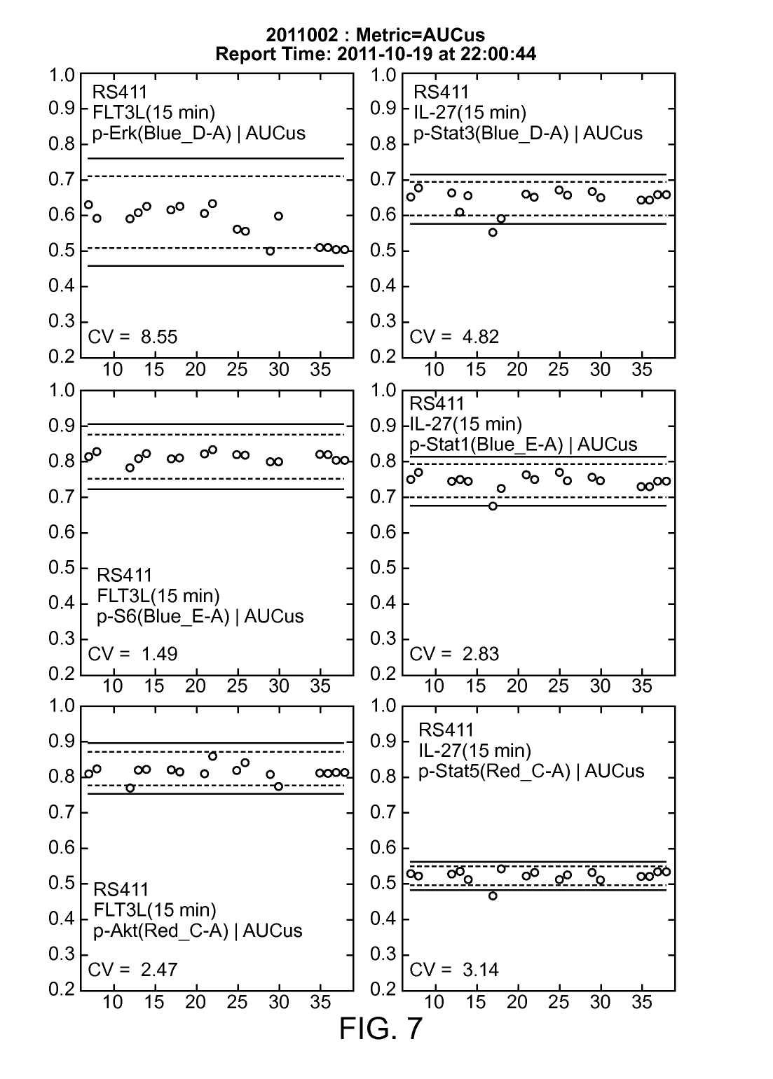

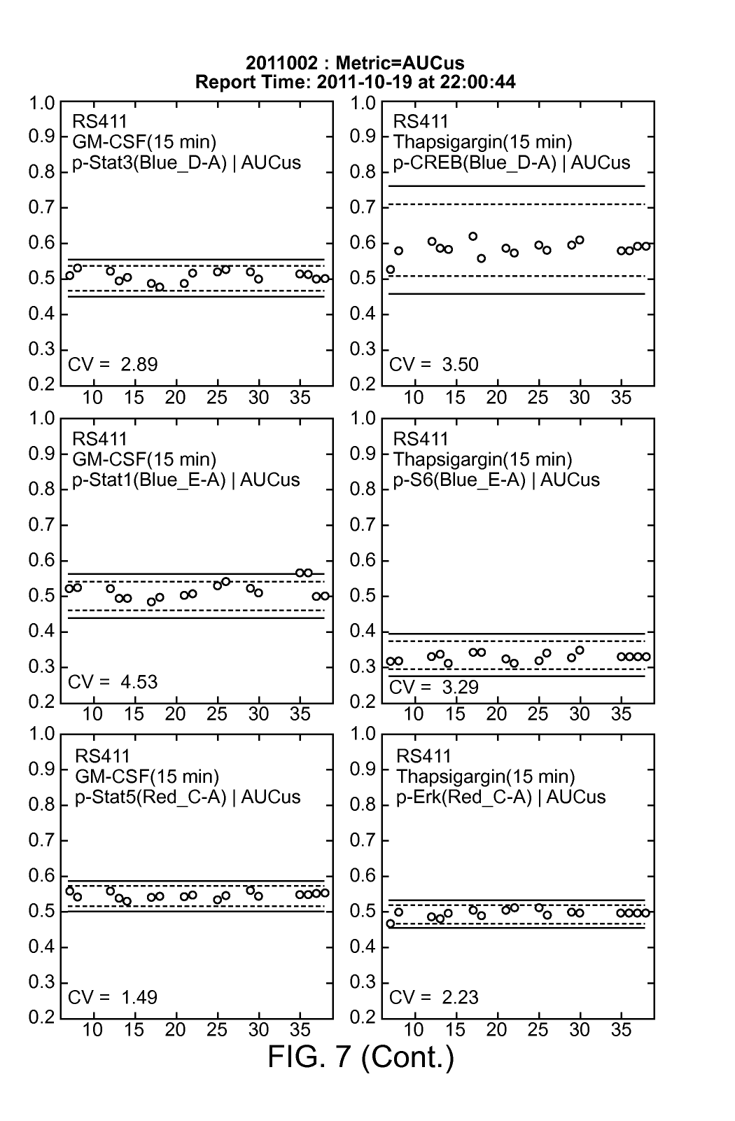

FIGS. 6 and 7 show CVs for cell lines RS;411 and GDM1 over another similar study.

### Example 3

### Assessment of Instrument Preformance

The present example shows another method to monitor assay and instrument variability by analyzing each microtiter plate. An assay similar to those shown above was run with the addition of beads designed to monitor the performance of the flow cytometers. See U.S. Pat. No. 8,187,885.

All instruments were configured and calibrated against quantitative Rainbow Control Particles (RCPs). Each microtiter plate contained one row of RCPs—calibrations performed plate-by-plate. Measurements such as the following were collected: 66 parameters collected—8 colors at 8 intensities, 2 scatter properties. Peaks numbered by intensity from low (Peak 1) to high (Peak 8). Peak 1 is below instrument noise level, is always excluded from analysis. Peak 2 shows the highest variance, as expected.

For plate and cytometer adjusted CVs, all values for peaks 2-8 on all channels are less than 3.3% and show that the instrument variation was approximately 1% across the beads on the plate. A total of 58 of 58 values for CVs across the plate are <2% (excluding Peak 1).

A total of 52 of 58 values for CVs across the cytometers (which include different processing days) are <2%.

### Example 4

### Quality Control of Flow Count Beads

The present example tests whether beads can be directly added to wells with the cell samples or if the presence of the beads would complicate that measurement. If so, then an indirect measurement in which beads would be used in non sample wells could be used.

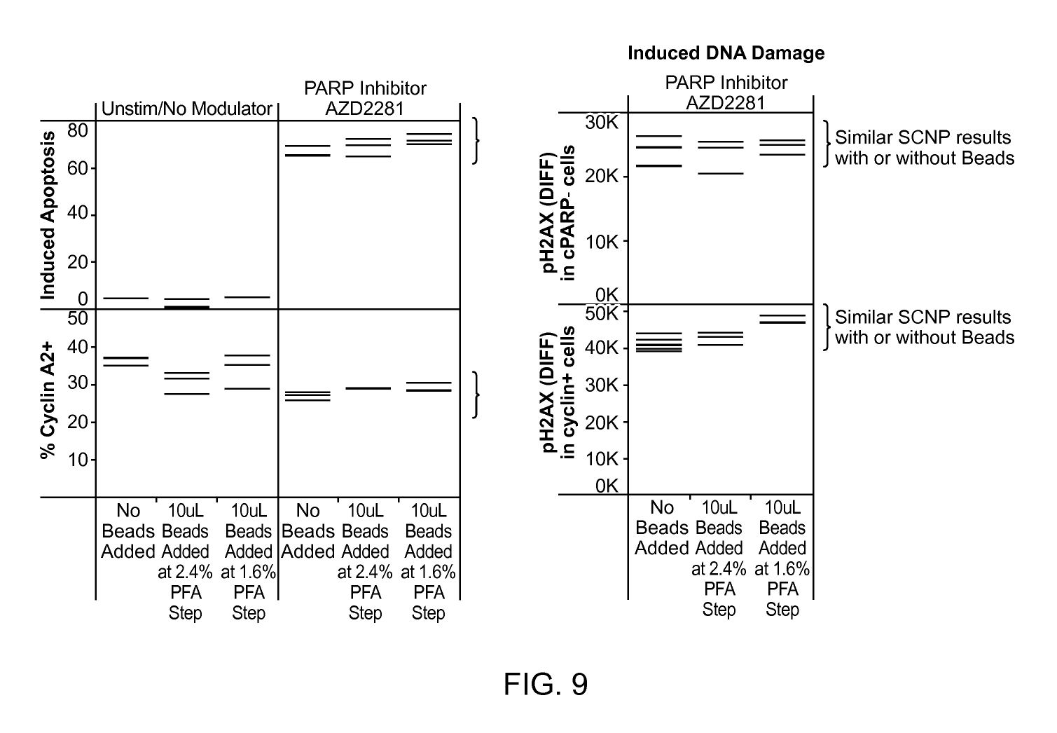

In the present example, GM13023 BRCA2 mutated cells were processed in a similar manner to that shown above. They were processed without beads and with beads in the same wells. See FIGS. 8 and 9 for the results which show that the resulting data are similar whether or not the beads are included with the cells. This shows that including the beads in the wells does not compromise the data obtained for the existing experiment and that adding beads can provide a direct quality control method.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A functional, quality control method for flow cytometry or mass spectrometry, comprising the steps of:
(a). Providing a microtiter plate;
(b). Distributing sample cells into wells of the microtiter plate;
(c). Distributing standard cells into wells of the microtiter plate;
(d). Distributing rainbow control particles (RCPs) into wells of the microtiter plate;
(e). Contacting the standard cells and sample cells with at least one modulator;
(f). Measuring one or more activatable elements and one or more surface markers in the standard cells by flow cytometry or mass spectrometry to create standard cell data;
(g). Measuring two activatable elements in the sample cells by flow cytometry or mass spectrometry to create test data;
(h). Measuring RCPs by flow cytometry or mass spectrometry to create RCP data;
(i). Comparing the standard cell data and RCP data to a preset range of acceptable values; and
(j). Normalizing or excluding the test data when the standard cell data or the RCP data is not within the preset range of acceptable values.

2. A functional, quality control method, for use in flow cytometry or mass spectrometry comprising the steps of:
(a). Measuring at least two activatable elements in standard cells to create standard cell data;
(b). Comparing the standard cell data to a preset range of acceptable values;
(c). Measuring at least two activatable elements in sample cells to create test data; and
(d). Excluding or normalizing test data when the standard cell data is not within the preset range of acceptable values.

3. The method of claim 2, further comprising the steps of:
(a). Providing at least one microtiter plate;
(b). Distributing live, sample and standard cells into wells of the microtiter plate;
(c). Measuring at least two activatable elements in the standard cells to create standard cell data;
(d). Measuring at least two activatable elements in the sample cells; and
(e). Excluding or normalizing test data when the standard cell data is within the preset range.

4. The method of claim 1, further comprising the steps of:
(a). Permeabilizing the cells with methanol using an instrument which has been prepared so that it dispenses an exact amount of methanol and has been charged with air or methanol when not in use; and
(b). Measuring at least two activatable elements using a flow cytometer.

5. The method in accordance with claim 2, wherein one or more processors executing computer readable code is used to execute a plurality of instructions to compare the standard cell data to a preset range of acceptable values.

6. The method in accordance with claim 2, wherein the standard cells are stable cell lines.

7. The method in accordance with claim 6, wherein the standard cells are stable cell lines GDM-1 or RS;411.

8. The method in accordance with claim 2, wherein the standard cells are measured prior to measuring each microtiter plate of the sample cells.

9. The method in accordance with claim 2, wherein the process used to measure the activatable elements in the standard cells and the sample cells comprises permeabilizing the cells with methanol with an instrument which has been prepared so that it dispenses an exact amount of methanol and has been charged with air or methanol when not in use.

10. The method in accordance with claim 9, wherein the instrument has a plurality of dispensing heads that dispense methanol into wells of microtiter plates and the dispensing heads have been charged with methanol using a bleeder valve.

11. The method in accordance with claim 9, further comprising adding beads to the wells containing the sample cells.

12. The method in accordance with claim 11, wherein the beads are used to normalize data across multiple wells.

13. The method in accordance with claim 1, wherein the RCPs are used in wells that have sample cells.

14. The method in accordance with claim 2, wherein the test data is adjusted based on normalization values.

15. The method in accordance with claim 2, further comprising providing a plurality of quality controls including the addition of beads, addition of stable cell lines, addition of stain controls, and the monitoring of cell surface or intracellular markers.

16. The method in accordance with claim 1, further comprising associating each step with the time it was performed.

17. The method in accordance with claim 2, further comprising placing quality control data into a database.

18. A kit to perform the method of claim 2, comprising two or more reagents, compounds or other devices selected from the group of: live cell lines, lyophilized cells, RCPs; and
three or more reagents selected from the group consisting of: antibodies directed to cell surface markers, antibodies directed to internal cell markers, modulators, buffers, fixatives, binding elements, and permeabilizers.

19. A kit to measure antibody addition in the method of claim 18 further comprising a cytometric capture array, buffers and reagents.

20. The method in accordance with claim 1, further comprising adding a cytometric bead array in the wells of the microtiter plate to measure modulators and antibodies.

21. The method in accordance with claim 2, wherein the standard cells are from healthy controls.

22. The method in accordance with claim 2, further comprising determining the cause of any results that not within acceptable values.

23. A functional, quality control method for use when analyzing samples with flow cytometry or mass spectrometry, comprising the steps of:
(a). Providing a holder having wells;
(b). Distributing sample cells into wells;
(c). Adding one or more reagents to wells, the reagents produce a consistent result under the same conditions used for the sample cells, the reagents include one or more of: standard cells, rainbow control particles (RCPs), and surface marker detection compounds;
(d). Contacting the sample cells and reagents with at least one modulator;
(e). Processing the reagents to obtain quality control data;
(f). Measuring at least two activatable elements in the sample cells to create test data;
(g). Comparing the quality control data to a preset range of acceptable values; and
(h). Analyzing the test data when the quality control data is within a preset range.
